(19) United States
(12) Patent Application Publication
(10) Pub. No.: US 2014/0005784 A1
(43) Pub. Date: Jan. 2, 2014

(54) TEXTURED PROSTHESIS WITH REDUCED CAPSULAR CONTRACTURE RESPONSE

(71) Applicant: Allergan, Inc., Irvine, CA (US)

(72) Inventors: Dennis E. Van Epps, Goleta, CA (US); Barbara Nofrey, Goleta, CA (US); Jordan Thompson, Chino Hills, CA (US)

(73) Assignee: Allergan, Inc., Irvine, CA (US)

(21) Appl. No.: 13/923,231

(22) Filed: Jun. 20, 2013

Related U.S. Application Data
(60) Provisional application No. 61/666,676, filed on Jun. 29, 2012.

(51) Int. Cl. A61F 2/12

(52) U.S. Cl. CPC A61F 2/12
USPC 623/8

(57) ABSTRACT

A method of augmenting or reconstructing a breast of a patient, the method comprising the step of implanting into the patient, a breast prosthesis, having surface characteristics directed at reducing collagen alignment in periprosthetic tissue.

This application claims priority to U.S. Provisional Patent Application No. 61/666,676, filed Jun. 29, 2012, the entire contents of which is hereby incorporated by reference.

### BACKGROUND

The present invention generally relates to medical implants and more specifically relates to materials suitable for implantation in a mammal which provide a reduced capsular contracture response around breast prostheses.

Prostheses or implants for augmentation and/or reconstruction of the human body are well known. Capsular contracture is a complication associated with surgical implantation of prostheses, particularly with soft implants, and even more particularly, though certainly not exclusively, with fluid-filled breast implants.

Capsular contracture is believed to be a result of the immune system response to the presence of a foreign material in the body. A normal response of the body to the presence of a newly implanted object, for example a breast implant, is to form periprosthetic tissue, sometimes in the form of a capsule containing collagen fibers around the implant. Capsular contracture occurs when the capsule begins to contract and squeeze the implant. This contracture can be discomforting or even extremely painful, and can cause distortion of the appearance of the augmented or reconstructed breast. The exact cause of contracture is not known. However, some factors that may influence contracture include bacterial contamination of the implant prior to placement, submuscular versus subglandular placement, and smooth surface implants versus textured surface implants, and bleeding or trauma to the area.

Surface texturing has been shown to reduce capsular contracture when implants are placed in the subglandular position compared to what are known as “smooth” surface implants. In other words, it is generally well known in the art that patients fitted with textured implants are less likely to exhibit contracture, relative to patients fitted with non-textured or smooth surface implants placed subglandularly. However, there is still a need for a textured implant that is specifically designed to encourage optimal tissue integration and potentially reduce capsule formation and collagen fiber alignment described herein.

### SUMMARY

Accordingly, materials including optimal surface textures are provided, such materials being useful as components of prostheses, for example, as components of breast implants, for example, soft, saline or silicone gel-filled breast implants.

The present materials are generally designed to achieve an optimal biological response in the patient after implantation thereof, for example, in the breast. The materials, sometimes herein referred to as “biomaterials”, are generally elastic and porous and comprise microstructures which contribute to healthy periprosthetic tissue ingrowth and reduced aligned fibrous capsule formation about a soft implant, resulting in reduced potential for capsular contracture.

The presently described materials enhance healthy periprosthetic tissue ingrowth, sometimes without the formation of an identifiable capsule. This integration of tissue into the textured surface of the materials may prevent, or substantially prevent, the formation of an organized, collagen-dense capsule around a soft tissue implant and disrupts the linearly aligned capsule/collagen fibers found with non-textured or lightly textured implants. Hence, contracture of any capsular tissue that may form around a soft tissue implant including the present microstructures may be avoided, or at least the potential therefor reduced.

In one aspect, the material is a soft, elastomeric open-cell material, for example, a foam-like material, having a microstructure discovered to enhance healthy tissue ingrowth.

The materials may be substantially non-biodegradable, and may comprise, for example, an elastomeric polymeric material, such as a medical grade silicone elastomer. In one embodiment, the materials consist essentially or entirely of a silicone elastomer.

In a specific embodiment, the porous materials, hereinafter sometimes referred to as, “microporous materials” are defined by highly interconnected cavities. The pore size, interconnectivity of pores and/or number of pore interconnections of the materials produce an optimal biological response, as defined elsewhere herein, when implanted in the human body, for example, when the material makes up a exterior surface or a covering of a breast prosthesis.

“Pore size”, as used herein, is defined as generally the diameter of a pore if spherical or the average of the major and minor axis of a pore if elliptical in shape.

In some embodiments, the microporous materials have a mean pore size of between about 30 μm to about 900 μm, for example, between about 300 microns and about 600 microns, for example, between about 350 μm to about 550 μm. In a particular embodiment, the mean pore size is about 450 μm. In another particular embodiment, the mean pore size is about 470 μm.

“Mean interconnection size”, as used herein, is defined as the approximate diameter of the opening between pores.

The microporous materials of the invention may have a mean interconnection size between the pores of about 50 μm to about 300 μm, for example, between about 150 microns to about 300 microns.

“Mean number of interconnections per pore”, as used herein, is defined as the average number of openings in each pore that connect to another pore.

In some embodiments, the materials have an a mean number of interconnections per pore of between about 2 to about 14, for example, about 3 to about 10, for example, about 9.

“Open cell”, as used herein, is defined as a characteristic of some of the materials of the present invention, in which pores or cells of the material are generally open to the surface of the material. In certain embodiments, the surface openness of the material is at least about 30%, for example, at least about 40%, for example, at least about 50%. This material can further be characterized in that the open cells are “interconnected” beneath the surface of the material, meaning that that pores or cells below surface-exposed pores or cells are in open communication, e.g. open connection, with each other. These open cell structures can be distinguished from “closed cell” structures in which each pore generally defines a discrete cavity, each cavity being completely surrounded by the solid material.

In one aspect, a material useful as a component of a mammary prosthesis is provided, the material comprising a textured surface. The textured surface has an open cell structure with the physical and morphological characteristics described herein. Mammary prostheses including these materials are also provided.

Methods for treating a patient in need of a mammary prosthesis are also provided.

In one aspect, the method comprises implanting the mammary prosthesis in the patient, the mammary prosthesis having a textured surface, wherein within a period of time of about six months to about one year or more after the implanting step, periprosthetic tissue formed in proximity to the textured surface is tissue that has reduced circumferential alignment of collagen fibers, improved adhesion, and will have a reduced risk of contracture, even for the useful life of the prosthesis. In certain embodiments, the method may be a breast augmentation procedure or a breast reconstruction procedure.

In another aspect, the method comprises implanting into the patient, a prosthesis comprising a open cell material having particular characteristics when tested in a test mammals. For example, in one embodiment, the material is characterized in that, when a one centimeter disc of a test material identical to the material is implanted subcutaneously in a Sprague Dawley rat, for example, using standard procedures, periprosthetic tissue forms adjacent the test material, and at least six weeks following the subcutaneous implantation, the periprosthetic tissue has a characteristic that a certain percentage of collagen fibers in the periprosthetic tissue are non-aligned with respect to a major planar surface of the test material.

For example, in one embodiment, the material comprises a nonresorbable, open cell, interconnected cellular material, which, after 6 weeks of being implanted in a rat, a collagen fiber alignment assay reveals that at least 22%, at of collagen fibers of the periprosthetic tissue are non-aligned with respect to a circumferential plane, or a major planar surface, defined by the test material.

In another embodiment, the material comprises an open interconnected cell material, which, after 6 weeks of being implanted in a rat, a collagen fiber alignment assay reveals that at least 25% of collagen fibers of the periprosthetic tissue are non-aligned with respect to a circumferential plane, or a major planar surface, defined by the test material.

In yet another embodiment, the material comprises an open interconnected cell material, which, after 6 weeks of being implanted in a rat, a collagen fiber alignment assay reveals that at least 50% of collagen fibers of the periprosthetic tissue are non-aligned with respect to a circumferential plane, or a major planar surface, defined by the test material.

In still yet another embodiment, the material comprises an open interconnected cell material, which, after 6 weeks of being implanted in a rat, a collagen fiber alignment assay reveals that at least 56% of collagen fibers of the periprosthetic tissue are non-aligned with respect to a circumferential plane, or a major planar surface, defined by the test material.

In some embodiments, the periprosthetic tissue, after six weeks of being so implanted in a rat, adheres to the test material with a force of at least 6 Newtons or greater. In yet another aspect of the invention, methods are provided for quantifying collagen fiber alignment in periprosthetic tissue in a mammal. In an exemplary embodiment, the method comprises obtaining a sample to be analyzed wherein the sample comprises periprosthetic tissue and adjacent material to be characterized that has been explanted from a mammal. At least one section, for example, two, three or more sections of the sample are then obtained using standard procedures wherein each section includes both periprosthetic tissue and at least a portion of the explanted adjacent material. Next, the section or sections are stained to reveal collagen fibers in the tissue under examination. The method further comprises providing a magnified image of the stained section and placing a reference vector on the magnified image, the reference vector being parallel to the major plane of the material at a tissue and material interface. In addition, a plurality of alignment vectors are placed on the image, for example, at least 10 or more, for example, about 25 alignment vectors, the alignment vectors being indicative of alignment of said collagen fibers revealed on the image. The method further comprises recording an angle of each of the alignment vectors with respect to the reference vector and calculating a variance of the recorded angles to thereby quantify a collagen fiber alignment of the sample of periprosthetic tissue. The method may further comprise grouping the recorded angles into bins, to create a histogram. The histogram is useful for making a determination of the degree or severity of collagen alignment in the periprosthetic tissue formed as a result of the implanted material. For example, the step of representing the alignment angles on a histogram may further include calculating the number of angles falling between about 80 degrees and 100 degrees, or between about 75 degrees and 105 degrees, which represent those collagen fibers that are most aligned with the major planar surface of the explanted material. In another aspect, the method may further comprise the step of performing a mathematical conversion on the alignment vectors to obtain a more quantitative analysis of the degree or severity of collagen alignment.

### BRIEF DESCRIPTION OF THE DRAWINGS

FIG. 1 graphically illustrates capsular contracture rates of textured implants of the prior art versus smooth implants, from various published studies.

FIG. 2A is a bar graph representing data of thickness of capsules from various materials, the values being normalized to “Textured 1”, and represented as a percent of “Textured 1”, a prior art textured material (BIOCELL®, Allergan, Inc., Irvine, Calif.), (as normalized mean±normalized standard deviation).

FIG. 2B is a bar graph representing disorganization of collagen in periprosthetic tissue from the various materials, normalized to “Textured 1” and represented as a percent of “Textured 1”, a prior art textured material (BIOCELL®, Allergan, Inc., Irvine, Calif.), (as normalized with a standard deviation with upper and lower bounds of confidence intervals). “Textured 5 & 6” represent open, interconnected cell structure.

FIGS. 2C-2D are graphical representations of angle measurements of collagen alignment in samples of tissue adjacent an implant.

FIGS. 2E-2F are diagrammatic representation vectors for a highly aligned tissue sample (FIG. 2E) and a highly unaligned tissue sample (FIG. 2F).

FIGS. 3A-3B show histogram representations of collagen fiber alignment assays described in this specification for different test materials.

FIG. 4 is a bar graph showing data from a tissue adhesion test of various materials. Results are shown as mean±standard deviation.

FIG. 5 is bar graph showing data of stiffness of capsule/ingrowth formed over various tissue expanders at time 0 and at 6 weeks (n=8). Results are shown as mean±standard deviation.

FIGS. 6-11 show images of a periprosthetic tissue adjacent various textured implants in animal studies.

FIGS. 12A-12D are images of materials in accordance with the invention, control and at selected time periods after implantation in a laboratory animal showing minimal or no loss of the structural integrity of the texture.

FIGS. 13A-13D are images of bioresorbable PRIOR ART materials, control and at selected time periods after implantation in a laboratory animal showing loss of structural integrity in a open cell bioresorbable matrix.

### DETAILED DESCRIPTION

Generally described herein are biocompatible materials which provide an optimal biological response of tissue adjacent the materials when the materials are implanted in a human body, for example, in a human breast. The materials are especially advantageous for use as components of soft gel-filled or saline filled breast implants, specifically as an exterior surface component of such breast implants.

Although the present specification primarily discloses the materials of the invention as components of breast implants, it can be appreciated that the materials of the invention may be suitable as components of other types of implantable devices, for example, but not limited to, other fillable or solid prostheses, pace makers, medical ports, catheters, dura mater substitutes, hernia meshes, cranial facial implants or other silicone or structural materials used as implants or prostheses.

In accordance with the present specification, an “optimal biological response” refers to a response of living tissue to the presence of the materials when the materials are implanted in a living body. Generally, the optimal biological response in terms of breast implants is sufficient tissue integration or adhesion to prevent rotation or shifting of the implant, and the development of relatively soft, thin periprosthetic tissue adjacent the implant, such periprosthetic tissue being unlike collagen-rich scar tissue or capsular tissue with highly aligned collagen fibers, and having a reduced likelihood of eliciting a contracture response. The periprosthetic tissue formed in response to the presence of the present implants will appear to have less linearly aligned collagen fibers with the ingrowth of tissue in and through the open interconnected pores of the texture, relative to capsular tissue that is more likely to be found around smooth or textured surfaces not composed of open, interconnected cell structures. Relative to capsular tissue that is typically found around closed-pore, or smooth implants, periprosthetic tissue formed in response to the present open cell materials when implanted will be more integrated with the implant, and may be more cellular, with collagen fibers being relatively less aligned with the major planar surfaces of the implant, (major planar surfaces of the implant are hereinafter sometimes referred to as “circumferential surfaces” of the implant).

For example, at least about 6 months, one year or preferably several years, after implantation of the presently described materials of the invention, periprosthetic tissue that can be characterized as capsular tissue will be effectively reduced or even absent. For example, less than about 60% or less than about 50% of the collagen fibers of the periprosthetic tissue formed will be parallel to the circumferential surfaces of the implant. Proper integration of tissue to the material, for example, by healthy tissue ingrowth, may also be considered a characteristic of an optimal biological response.

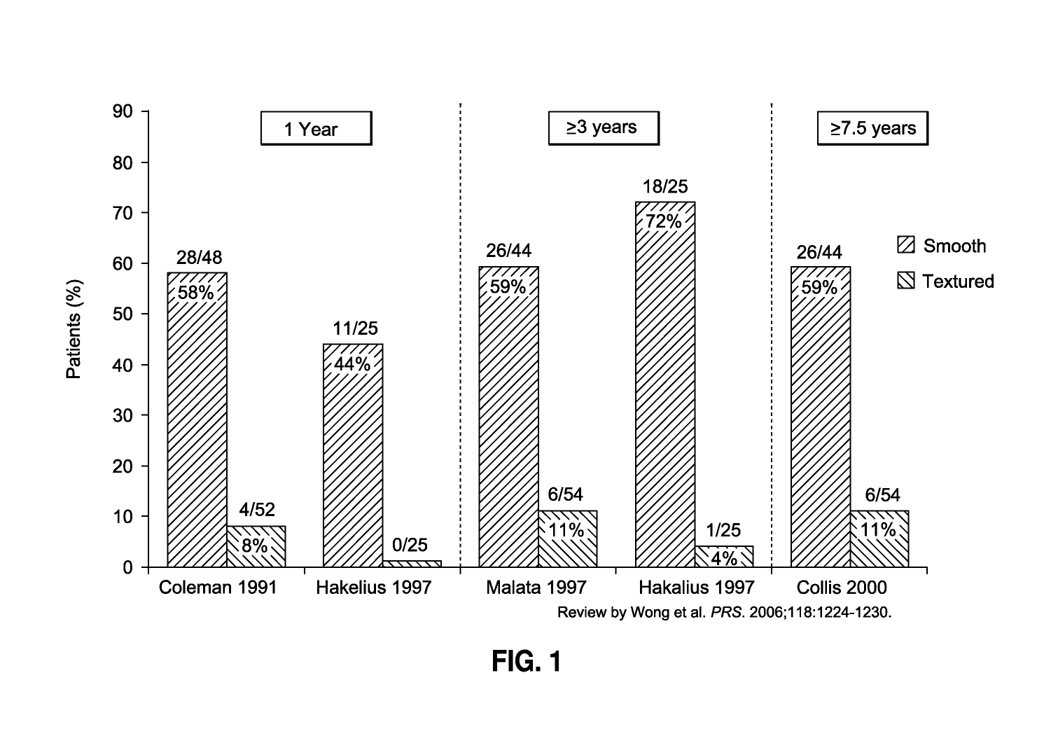

As mentioned hereinabove, it is believed that the use of textured implants reduces the potential for capsular contracture of surrounding tissues, relative to the use of so-called smooth surfaced implants. FIG. 1 graphically illustrates this conventional knowledge based on various studies which indicated the reduction in occurrence of contracture in subglandular placed textured implants versus smooth implants. FIG. 1 shows a comparison of percentages of patients with subglandular implants, who experienced capsular contracture, relative to whether they received smooth implants or textured implants. Prior to the present invention, is has been generally unknown what particular texture characteristics produce the lowest incidence of contracture.

In accordance with the present invention, textured material geometries have been discovered which produce an optimal biological response, a loss of alignment of the collagen fibers at the surface of the implant and a reduction or elimination of capsular contracture.

The materials described herein can achieve an optimal biological response, for example, reduced capsule formation and proper or elevated attachment to surrounding tissues, when compared to textured materials commonly used in the art.

In accordance with one aspect of the invention, the materials are highly porous materials, having highly interconnected cells and cavities. These materials are considered to be “open-cell” materials, in that individual cavities or cells have passages to adjacent cavities or cells, rather than being isolated or closed off from one another by the solid materials. The open-cell structure of the present materials has been discovered to facilitate healthy tissue ingrowth and integration of tissue into the materials. The ingrowth of tissue may also reduce the circumferential alignment of collagen fibers with respect to the surface of the implant, leading to a reduced likelihood of contracture.

Periprosthetic tissue adjacent an implant has no defined thickness or width; it is simply the tissue that forms adjacent an implant. For purposes of the present disclosure, periprosthetic tissue in a human being has a mean thickness of about 20 microns to 1000 microns, with the thinnest being 20 microns and the thickest being 1000 microns (1 mm). Average human thickness of periprosthetic tissue is about 370 micrometers with a standard deviation of about 210 micrometers. This thickness varies with the time the implant has been in the body and the degree of capsule contracture.

In an exemplary embodiment, the material may be a microporous material having generally uniformly sized pores, with a mean pore size from about 30 μm to about 900 μm, for example, between about 400 μm to about 550 μm, or about 410 μm to about 530 μm, or about 450 μm to about 490 μm, for example, about 470 μm. The pores may have a mean interconnection size between pores of about 150 μm to about 300 μm, or about 175 μm to about 270 μm, or about 180 μm to about 240 μm. In one embodiment, the mean interconnection size is about 210 μm. Interconnections per pore may be between about 2 to about 14.

Preferably, the materials are highly porous, having a porosity (open space) of at least about 50% to about 98% or greater. In some embodiments, such porous materials are provided which have a porosity of about 80% to about 88%, for example, about 83% to about 85%.

Ranges of suitable values for porosity of materials of the invention.

| Characteristic | Range of values |
|---|---|
| Thickness (mm) | 0.8 to 2.9 |
| Pore size (um) | 30 to 900 |
| Interconnections | 2 to 14 |
| Porosity % open space | 50 to 98 |
| Interconnection pore size (um) | 50 to 300 |

The materials of the present invention can be made by any suitable manufacturing process that will produce a porous material having the desired architecture for producing an optimal biological response as defined herein. Suitable methods of making these materials are described in, for example, commonly owned U.S. patent application Ser. Nos. 12/261,939, filed Oct. 30, 2009; 12/897,498, filed Oct. 4, 2010; 12/778,813, filed May 12, 2010; 13/160,325, filed Jun. 14, 2011; 13/015,309, filed Jan. 27, 2011; 13/104,893, filed May 10, 2011; 13/104,888, filed May 10, 2011; 13/104,820, filed May 10, 2011; 13/245,518, filed Sep. 26, 2011; 13/021,615, filed Feb. 4, 2011; 13/093,505, filed Apr. 25, 2011; 13/104,395, filed May 10, 2011; 13/314,116, filed Dec. 7, 2011; 13/104,811, filed May 10, 2011; 13/247,835, filed Sep. 28, 2011; 13/246,568, filed Sep. 27, 2011; 13/297,120, filed Nov. 15, 2011; 13/247,535, filed Sep. 28, 2011; and 13/213,925, filed Aug. 19, 2011, the entire disclosure of each of these applications being incorporated herein by this reference.

In one aspect of the invention, the textured materials are non-bioresorbable and may be substantially entirely silicone. The microstructure of the texture thus remains substantially entirely unchanged for the useful life of the implant, for example, for at least 6 month, for at least one year, for at least 6 years or more. In other words, the cavity struts forming the pores of the texture do not change over time. This is distinguished from certain degradable textured implants which over time, become smoother, or substantially less textured, and potentially more prone to inducing formation of aligned collagen tissue and contracture. This is a substantial advantage of some of the textured materials of the present invention relative to certain other textured materials, such as porous polyurethanes, that may have a similar microstructure when first implanted in the breast but which degrade and change over time. This characteristic of prior art polyurethane-covered textured breast implants may be more clearly understood with reference to the publication “How Texture-Inducing Contraction Vectors Affect the Fibrous Capsule Shrinkage Around Breast Implants?”, Abramo, et al., Aest Plast Surg (2010) 34; 555-560.

In some embodiments, the presently silicone textured surfaces are a unitary molded part of the breast implant shell. In other embodiments, the materials of the invention are coupled, by means of a suitable coupling technique, material or method, to an outer surface of an implantable device. In some embodiments, the materials are applied to a breast implant, to cover or coat the entire outer surface of a shell of a fillable breast implant. In other embodiments, the materials are applied to less than the full outer surface of a breast implant, for example, to only a portion of a shell of a fillable breast implant. For example, only the front of the shell may be covered or coated with the materials of the invention, or only the back of the shell may be covered or coated with the materials of the invention, or only about 20%, about 30%, about 40%, about 50%, about 60%, about 70% about 80% or about 90% of the shell may be covered or coated with the materials. In other embodiments, substantially all of the shell is covered or coated with the materials of the invention.

For example, the materials may be bonded to a surface of a smooth breast implant by use of a room temperature vulcanizing silicone (RTV) or high temperature vulcanizing (HTV) silicone. The bonding substance can be applied to the materials using any method known in the art, for example, brushing, spraying, dipping, curtain coating, vapor deposition methods, casting methods, injection molding and the like. The bonding substance can be cured using heat or any other means of aiding in curing known in the art.

After the material has been adhered to the surface of an implantable shell, extra portions of the material can be removed from the shell by trimming.

Materials as described herein can be laminated onto a smooth breast implant shell using silicone adhesive. The lamination can be done while the shell is cured or uncured and still on its molding mandrel, or alternatively, on a finished, cured shell. For example, a dispersion of HTV silicone may be used as the adhesive between the implant and material sheets. In the process, the first material sheet is coated with a thin layer of HTV silicone and then placed in the bottom cavity. The smooth implant is then placed on top of the material sheet in the cavity. The second foam sheet is coated with a thin layer of HTV silicone and applied on top of the smooth implant. The top piece of the cavity is then fixed in place pressing the two material sheets together creating a uniform interface. The silicone adhesive is allowed to cure and then the excess foam is cut off creating a uniform seam around the implant.

Another exemplary process involves laminating the material onto a smooth implant still on a mandrel. In this process a HTV silicone is used as the adhesive between the implant and the material sheets. A first material sheet is coated with a thin layer of HTV silicone and then draped over the smooth implant on the mandrel in such a way that there are no wrinkles on the top surface. After this has cured, another coating of HTV silicone is applied and the material is stretched up to cover part of the back of the implant. The smooth implant is then taken off the mandrel and the excess material is removed. A smaller circle is cut out of a material sheet to fit the back of the implant. A thin layer of HTV silicone is applied to the small circle of material and the circle is attached and allowed to cure.

In another embodiment, a bonding surface is applied to the implant by dipping the implant into HTV silicone and then the material is applied onto the implant. The HTV silicone can be applied to the implant using any technique known to those skilled in the art, for example, by spraying curtain coating, and the like.

In yet another embodiment, the implantable shell is coated with an emulsion including an agitated mixture of a first organic solvent and at least one extractable agent, and a second organic solvent and at least one silicone matrix agent. The emulsion can also be applied to the implantable shell. A common method used to coat an implantable shell is to first form the shell itself on a mandrel using a dipping technique and then after the shell is formed, to dip that formed shell into a composition as described herein. The emulsion is then allowed to cure on the implantable shell thereby forming an open-cell material. Extractable materials can then be removed from the material using various drying and/or leaching techniques known in the art. In one example embodiment, the curing step optionally includes heating.

The extractable agent, or removable polymer may be, for example, a water soluble material dispersed throughout the curable elastomer. Typical extractable agents or leachable materials may comprise, for example, polyethylene glycol (PEG, also known as polyoxyethylene), polyalkylene oxides including polyethylene oxide and polyethylene oxide/polypropylene oxide copolymers (also known as poloxamers), polyhydroxyethylmethacrylate, polyvinylpyrrolidone, polyacrylamide, or other substituted polyolefins and their copolymers, polylactides, polyglycolides, or other polyesters, polyanhydrides, polyorthoesters and their copolymers, proteins including albumin, peptides, liposomes, cationic lipids, ionic or nonionic detergents, salts including potassium chloride, sodium chloride and calcium chloride, sugars including galactose, glucose and sucrose, polysaccharides including soluble celluloses, heparin, cyclodextrins and dextran, and blends of the same.

The solvent component of the composition can include a solvent selected from the group consisting of xylene, pentane, hexane, dichloromethane (DCM), dimethyl sulfoxide, dioxane, NMP, DMAc, and combinations thereof or any other protic or aprotic solvent or combinations thereof.

In one embodiment described herein are implantable composite members having an external surface at least a portion of which is covered by a material as described herein which can attain an optimal biological response. The materials can impart the optimal biological response to the implantable member. The implantable composite members are made by first providing an implantable member (e.g., an implantable shell or implantable medical device) and providing a material as described herein. Next, a bonding substance is applied to a chosen material, thereby forming a bondable material. The bonding substance will act as a means for attaching the material to the implantable member. The bondable material is then applied to at least a portion of the implantable member and the bonding substance is cured.

A method has been described for creating an outer layer having at least one of the materials described herein. Further, the method can be applied to create a medical implant with an external surface layer of a foam and/or felt as described herein for use in creating strips having a textured surface for control of scar or capsule formation, or to improve a process for making mammary prostheses. The product made by this method has utility in preventing capsular contraction, in preventing or controlling capsule or scar formation, and in anchoring medical implants.

It is often important to anchor medical implants to prevent implant movement, displacement or rotation. Mammary prostheses are one example of implants that are preferentially anchored. Facial implants are another example of implants that can be anchored. With facial implants, for example, it is important that they be anchored securely against movement because of their prominent location. Providing such implants with foam or felt surface made in accordance with the present description is an advantageous way to ensure that they will be anchored securely as tissue ingrowth once implanted will prevent their migration.

A porous material comprising an elastomer matrix includes pores having a shape sufficient to allow tissue growth into the array of interconnected pores. As such, the pore shape should support aspects of tissue growth such as, e.g., cell migration, cell proliferation, cell differentiation, nutrient exchange, and/or waste removal. Any pore shape is useful with the proviso that the pore shape is sufficient to allow tissue growth into the array of interconnected pores. Useful pore shapes include, without limitation, roughly spherical, perfectly spherical, dodecahedrons (such as pentagonal dodecahedrons), and ellipsoids.

A porous material comprising an elastomer matrix includes pores having a roundness sufficient to allow tissue growth into the array of interconnected pores. As such, the pore roundness should support aspects of tissue growth such as, e.g., cell migration, cell proliferation, cell differentiation, nutrient exchange, and/or waste removal. As used herein, “roundness” is defined as (6×V)/(π×D³), where V is the volume and D is the diameter. Any pore roundness is useful with the proviso that the pore roundness is sufficient to allow tissue growth into the array of interconnected pores.

A porous material comprising an elastomer matrix is formed in such a manner that substantially all the pores in the elastomer matrix have a similar diameter. As used herein, the term “substantially,” when used to describe pores, refers to at least 90% of the pores within the elastomer matrix such as, e.g., at least 95% or at least 97% of the pores. As used herein, the term “similar diameter,” when used to describe pores, refers to a difference in the diameters of the two pores that is less than about 20% of the larger diameter. As used herein, the term “diameter,” when used to describe pores, refers to the longest line segment that can be drawn that connects two points within the pore, regardless of whether the line passes outside the boundary of the pore. Any pore diameter is useful with the proviso that the pore diameter is sufficient to allow tissue growth into the porous material. As such, the pore diameter size should support aspects of tissue growth such as, e.g., cell migration, cell proliferation, cell differentiation, nutrient exchange, and/or waste removal.

A porous material comprising an elastomer matrix is formed in such a manner that the diameter of the connections between pores is sufficient to allow tissue growth into the array of interconnected pores. As such, the diameter of the connections between pores should support aspects of tissue growth such as, e.g., cell migration, cell proliferation, cell differentiation, nutrient exchange, and/or waste removal. As used herein, the term “diameter,” when describing the connection between pores, refers to the diameter of the cross-section of the connection between two pores in the plane normal to the line connecting the centroids of the two pores, where the plane is chosen so that the area of the cross-section of the connection is at its minimum value. As used herein, the term “diameter of a cross-section of a connection” refers to the average length of a straight line segment that passes through the center, or centroid (in the case of a connection having a cross-section that lacks a center), of the cross-section of a connection and terminates at the periphery of the cross-section. As used herein, the term “substantially,” when used to describe the connections between pores refers to at least 90% of the connections made between each pore comprising the elastomer matrix, such as, e.g., at least 95% or at least 97% of the connections.

Thus, in an embodiment, a porous material comprising an elastomer matrix includes pores having a roundness sufficient to allow tissue growth into the array of interconnected pores. In aspects of this embodiment, a porous material comprising an elastomer matrix includes pores having a roundness of, e.g., about 0.1, about 0.2, about 0.3, about 0.4, about 0.5, about 0.6, about 0.7, about 0.8, about 0.9, or about 1.0. In other aspects of this embodiment, a porous material comprising an elastomer matrix includes pores having a roundness of, e.g., at least 0.1, at least 0.2, at least 0.3, at least 0.4, at least 0.5, at least 0.6, at least 0.7, at least 0.8, at least 0.9, or at least 1.0. In yet other aspects of this embodiment, a porous material comprising an elastomer matrix includes pores having a roundness of, e.g., at most 0.1, at most 0.2, at most 0.3, at most 0.4, at most 0.5, at most 0.6, at most 0.7, at most 0.8, at most 0.9, or at most 1.0. In still other aspects of this embodiment, a porous material comprising an elastomer matrix includes pores having a roundness of, e.g., about 0.1 to about 1.0, about 0.2 to about 1.0, about 0.3 to about 1.0, about 0.4 to about 1.0, about 0.5 to about 1.0, about 0.6 to about 1.0, about 0.7 to about 1.0, about 0.8 to about 1.0, about 0.9 to about 1.0, about 0.1 to about 0.9, about 0.2 to about 0.9, about 0.3 to about 0.9, about 0.4 to about 0.9, about 0.5 to about 0.9, about 0.6 to about 0.9, about 0.7 to about 0.9, about 0.8 to about 0.9, about 0.1 to about 0.8, about 0.2 to about 0.8, about 0.3 to about 0.8, about 0.4 to about 0.8, about 0.5 to about 0.8, about 0.6 to about 0.8, about 0.7 to about 0.8, about 0.1 to about 0.7, about 0.2 to about 0.7, about 0.3 to about 0.7, about 0.4 to about 0.7, about 0.5 to about 0.7, about 0.6 to about 0.7, about 0.1 to about 0.6, about 0.2 to about 0.6, about 0.3 to about 0.6, about 0.4 to about 0.6, about 0.5 to about 0.6, about 0.1 to about 0.5, about 0.2 to about 0.5, about 0.3 to about 0.5, or about 0.4 to about 0.5.

In another embodiment, substantially all pores within a porous material comprising an elastomer matrix have a similar diameter. In aspects of this embodiment, at least 90% of all pores within a porous material comprising an elastomer matrix have a similar diameter, at least 95% of all pores within a porous material comprising an elastomer matrix have a similar diameter, or at least 97% of all pores within a porous material comprising an elastomer matrix have a similar diameter. In another aspect of this embodiment, difference in the diameters of two pores is, e.g., less than about 20% of the larger diameter, less than about 15% of the larger diameter, less than about 10% of the larger diameter, or less than about 5% of the larger diameter.

In another embodiment, a porous material comprising an elastomer matrix includes pores having a mean diameter sufficient to allow tissue growth into the array of interconnected pores. In aspects of this embodiment, a porous material comprising an elastomer matrix includes pores having mean pore diameter of, e.g., about 50 μm, about 75 μm, about 100 μm, about 150 μm, about 200 μm, about 250 μm, about 300 μm, about 350 μm, about 400 μm, about 450 μm, or about 500 μm. In other aspects, a porous material comprising an elastomer matrix includes pores having mean pore diameter of, e.g., about 500 μm, about 600 μm, about 700 μm, about 800 μm, about 900 μm, about 1000 μm, about 1500 μm, about 2000 μm, about 2500 μm, or about 3000 μm. In yet other aspects of this embodiment, a porous material comprising an elastomer matrix includes pores having mean pore diameter of, e.g., at least 50 μm, at least 75 μm, at least 100 μm, at least 150 μm, at least 200 μm, at least 250 μm, at least 300 μm, at least 350 μm, at least 400 μm, at least 450 μm, or at least 500 μm. In still other aspects, a porous material comprising an elastomer matrix includes pores having mean pore diameter of, e.g., at least 500 μm, at least 600 μm, at least 700 μm, at least 800 μm, at least 900 μm, at least 1000 μm, at least 1500 μm, at least 2000 μm, at least 2500 μm, or at least 3000 μm. In further aspects of this embodiment, a porous material comprising an elastomer matrix includes pores having mean pore diameter of, e.g., at most 50 μm, at most 75 μm, at most 100 μm, at most 150 μm, at most 200 μm, at most 250 μm, at most 300 μm, at most 350 μm, at most 400 μm, at most 450 μm, or at most 500 μm. In yet further aspects of this embodiment, a porous material comprising an elastomer matrix includes pores having mean pore diameter of, e.g., at most 500 μm, at most 600 μm, at most 700 μm, at most 800 μm, at most 900 μm, at most 1000 μm, at most 1500 μm, at most 2000 μm, at most 2500 μm, or at most 3000 μm. In still further aspects of this embodiment, a porous material comprising an elastomer matrix includes pores having mean pore diameter in a range from, e.g., about 300 μm to about 600 μm, about 200 μm to about 700 μm, about 100 μm to about 800 μm, about 500 μm to about 800 μm, about 50 μm to about 500 μm, about 75 μm to about 500 μm, about 100 μm to about 500 μm, about 200 μm to about 500 μm, about 300 μm to about 500 μm, about 50 μm to about 1000 μm, about 75 μm to about 1000 μm, about 100 μm to about 1000 μm, about 200 μm to about 1000 μm, about 300 μm to about 1000 μm, about 50 μm to about 1000 μm, about 75 μm to about 3000 μm, about 100 μm to about 3000 μm, about 200 μm to about 3000 μm, or about 300 μm to about 3000 μm.

In another embodiment, a porous material comprising an elastomer matrix includes pores having a mean elastomer strut thickness sufficient to allow tissue growth into the array of interconnected pores. In aspects of this embodiment, a porous material comprising an elastomer matrix includes pores having a mean elastomer strut thickness of, e.g., about 10 μm, about 20 μm, about 30 μm, about 40 μm, about 50 μm, about 60 μm, about 70 μm, about 80 μm, about 90 μm, about 100 μm, about 110 μm, about 120 μm, about 130 μm, about 140 μm, about 150 μm, about 160 μm, about 170 μm, about 180 μm, about 190 μm, or about 200 μm. In other aspects of this embodiment, a porous material comprising an elastomer matrix includes pores having a mean elastomer strut thickness of, e.g., at least 10 μm, at least 20 μm, at least 30 μm, at least 40 μm, at least 50 μm, at least 60 μm, at least 70 μm, at least 80 μm, at least 90 μm, at least 100 μm, at least 110 μm, at least 120 μm, at least 130 μm, at least 140 μm, at least 150 μm, at least 160 μm, at least 170 μm, at least 180 μm, at least 190 μm, or at least 200 μm. In yet other aspects of this embodiment, a porous material comprising an elastomer matrix includes pores having a mean elastomer strut thickness of, e.g., at most 10 μm, at most 20 μm, at most 30 μm, at most 40 μm, at most 50 μm, at most 60 μm, at most 70 μm, at most 80 μm, at most 90 μm, at most 100 μm, at most 110 μm, at most 120 μm, at most 130 μm, at most 140 μm, at most 150 μm, at most 160 μm, at most 170 μm, at most 180 μm, at most 190 μm, or at most 200 μm. In still aspects of this embodiment, a porous material comprising an elastomer matrix includes pores having a mean elastomer strut thickness of, e.g., about 50 μm to about 110 μm, about 50 μm to about 120 μm, about 50 μm to about 130 μm, about 50 μm to about 140 μm, about 50 μm to about 150 μm, about 60 μm to about 110 μm, about 60 μm to about 120 μm, about 60 μm to about 130 μm, about 60 μm to about 140 μm, about 70 μm to about 110 μm, about 70 μm to about 120 μm, about 70 μm to about 130 μm, or about 70 μm to about 140 μm.

In another embodiment, a porous material comprising an elastomer matrix includes pores connected to a plurality of other pores. In aspects of this embodiment, a porous material comprising an elastomer matrix comprises a mean pore connectivity, e.g., about two other pores, about three other pores, about four other pores, about five other pores, about six other pores, about seven other pores, about eight other pores, about nine other pores, about ten other pores, about 11 other pores, or about 12 other pores. In other aspects of this embodiment, a porous material comprising an elastomer matrix comprises a mean pore connectivity, e.g., at least two other pores, at least three other pores, at least four other pores, at least five other pores, at least six other pores, at least seven other pores, at least eight other pores, at least nine other pores, at least ten other pores, at least 11 other pores, or at least 12 other pores. In yet other aspects of this embodiment, a porous material comprising an elastomer matrix comprises a mean pore connectivity, e.g., at most two other pores, at least most other pores, at least most other pores, at least most other pores, at most six other pores, at most seven other pores, at most eight other pores, at most nine other pores, at most ten other pores, at most 11 other pores, or at most 12 other pores.

In still other aspects of this embodiment, a porous material comprising an elastomer matrix includes pores connected to, e.g., about two other pores to about 12 other pores, about two other pores to about 11 other pores, about two other pores to about ten other pores, about two other pores to about nine other pores, about two other pores to about eight other pores, about two other pores to about seven other pores, about two other pores to about six other pores, about two other pores to about five other pores, about three other pores to about 12 other pores, about three other pores to about 11 other pores, about three other pores to about ten other pores, about three other pores to about nine other pores, about three other pores to about eight other pores, about three other pores to about seven other pores, about three other pores to about six other pores, about three other pores to about five other pores, about four other pores to about 12 other pores, about four other pores to about 11 other pores, about four other pores to about ten other pores, about four other pores to about nine other pores, about four other pores to about eight other pores, about four other pores to about seven other pores, about four other pores to about six other pores, about four other pores to about five other pores, about five other pores to about 12 other pores, about five other pores to about 11 other pores, about five other pores to about ten other pores, about five other pores to about nine other pores, about five other pores to about eight other pores, about five other pores to about seven other pores, or about five other pores to about six other pores.

In another embodiment, a porous material comprising an elastomer matrix includes a surface openness sufficient to allow tissue growth into the array of interconnected pores. Surface openness, or first level openness, refers to the percentage area that the pores at the surface of a porous material are exposed to the surroundings. Surface openness may be determined by examining a top view image of a porous material. In aspects of this embodiment, a porous material comprising an elastomer matrix includes a surface openness of, e.g., about 50%, about 55%, about 60%, about 65%, about 70%, about 75%, about 80%, about 85%, about 90%, about 93%, about 95%, about 97%, or about 100%. In other aspects of this embodiment, a porous material comprising an elastomer matrix includes a surface openness of, e.g., at least 50%, at least 55%, at least 60%, at least 65%, at least 70%, at least 75%, at least 80%, at least 85%, at least 90%, at least 93%, at least 95%, at least 97%, or at least 100%. In yet other aspects of this embodiment, a porous material comprising an elastomer matrix includes a surface openness of, e.g., about 50% to about 100%, about 55% to about 100%, about 60% to about 100%, about 65% to about 100%, about 70% to about 100%, about 75% to about 100%, about 80% to about 100%, or about 85% to about 100%.

In another embodiment, a porous material is provided comprising an elastomer matrix includes an interconnectivity between pores sufficient to allow tissue growth into the array of interconnected pores. Interconnectivity, or second level openness, may be determined by measuring the area of visible openings or interconnections within each pore or surface opening from a top view image of a porous material and relating that area to the total area of the analyzed image. In aspects of this embodiment, a porous material comprising an elastomer matrix includes an interconnectivity between pores of, e.g., about 8%, about 9%, about 10%, about 11%, about 12%, about 13%, about 14%, about 15%, about 16%, about 17%, about 18%, about 19%, or about 20%. In other aspects of this embodiment, a porous material comprising an elastomer matrix includes an interconnectivity between pores of, e.g., at least 8%, at least 9%, at least 10%, at least 11%, at least 12%, at least 13%, at least 14%, at least 15%, at least 16%, at least 17%, at least 18%, at least 19%, or at least 20%. In yet other aspects of this embodiment, a porous material comprising an elastomer matrix includes an interconnectivity between pores of, e.g., about 8% to about 20%, about 9% to about 20%, about 10% to about 20%, about 11% to about 20%, about 12% to about 20%, about 13% to about 20%, about 14% to about 20%, or about 15% to about 20%.

In another embodiment, a porous material comprising an elastomer matrix includes pores where the diameter of the connections between pores is sufficient to allow tissue growth into the array of interconnected pores. In aspects of this embodiment, a porous material comprising an elastomer matrix includes pores where the diameter of the connections between pores is, e.g., about 10% the mean pore diameter, about 20% the mean pore diameter, about 30% the mean pore diameter, about 40% the mean pore diameter, about 50% the mean pore diameter, about 60% the mean pore diameter, about 70% the mean pore diameter, about 80% the mean pore diameter, or about 90% the mean pore diameter. In other aspects of this embodiment, a porous material comprising an elastomer matrix includes pores where the diameter of the connections between pores is, e.g., at least 10% the mean pore diameter, at least 20% the mean pore diameter, at least 30% the mean pore diameter, at least 40% the mean pore diameter, at least 50% the mean pore diameter, at least 60% the mean pore diameter, at least 70% the mean pore diameter, at least 80% the mean pore diameter, or at least 90% the mean pore diameter. In yet other aspects of this embodiment, a porous material comprising an elastomer matrix includes pores where the diameter of the connections between pores is, e.g., at most 10% the mean pore diameter, at most 20% the mean pore diameter, at most 30% the mean pore diameter, at most 40% the mean pore diameter, at most 50% the mean pore diameter, at most 60% the mean pore diameter, at most 70% the mean pore diameter, at most 80% the mean pore diameter, or at most 90% the mean pore diameter.

In still other aspects of this embodiment, a porous material comprising an elastomer matrix includes pores where the diameter of the connections between pores is, e.g., about 10% to about 90% the mean pore diameter, about 15% to about 90% the mean pore diameter, about 20% to about 90% the mean pore diameter, about 25% to about 90% the mean pore diameter, about 30% to about 90% the mean pore diameter, about 35% to about 90% the mean pore diameter, about 40% to about 90% the mean pore diameter, about 10% to about 80% the mean pore diameter, about 15% to about 80% the mean pore diameter, about 20% to about 80% the mean pore diameter, about 25% to about 80% the mean pore diameter, about 30% to about 80% the mean pore diameter, about 35% to about 80% the mean pore diameter, about 40% to about 80% the mean pore diameter, about 10% to about 70% the mean pore diameter, about 15% to about 70% the mean pore diameter, about 20% to about 70% the mean pore diameter, about 25% to about 70% the mean pore diameter, about 30% to about 70% the mean pore diameter, about 35% to about 70% the mean pore diameter, about 40% to about 70% the mean pore diameter, about 10% to about 60% the mean pore diameter, about 15% to about 60% the mean pore diameter, about 20% to about 60% the mean pore diameter, about 25% to about 60% the mean pore diameter, about 30% to about 60% the mean pore diameter, about 35% to about 60% the mean pore diameter, about 40% to about 60% the mean pore diameter, about 10% to about 50% the mean pore diameter, about 15% to about 50% the mean pore diameter, about 20% to about 50% the mean pore diameter, about 25% to about 50% the mean pore diameter, about 30% to about 50% the mean pore diameter, about 10% to about 40% the mean pore diameter, about 15% to about 40% the mean pore diameter, about 20% to about 40% the mean pore diameter, about 25% to about 40% the mean pore diameter, or about 30% to about 40% the mean pore diameter.

The present specification discloses, in part, a porous material comprising an elastomer matrix defining an array of interconnected pores having a porosity that is sufficient to allow tissue growth into the array of interconnected pores as disclosed herein. As such, the porosity should support aspects of tissue growth such as, e.g., cell migration, cell proliferation, cell differentiation, nutrient exchange, and/or waste removal. As used herein, the term “porosity” refers to the amount of void space in a porous material comprising an elastomer matrix. As such, the total volume of a porous material comprising an elastomer matrix disclosed herein is based upon the elastomer space and the void space.

Thus, in an embodiment, a porous material comprising an elastomer matrix defining an array of interconnected pores has a porosity sufficient to allow tissue growth into the array of interconnected pores. In aspects of this embodiment, a porous material comprising an elastomer matrix comprises a porosity of, e.g., about 40% of the total volume of an elastomer matrix, about 50% of the total volume of an elastomer matrix, about 60% of the total volume of an elastomer matrix, about 70% of the total volume of an elastomer matrix, about 80% of the total volume of an elastomer matrix, about 90% of the total volume of an elastomer matrix, about 95% of the total volume of an elastomer matrix, or about 97% of the total volume of an elastomer matrix. In other aspects of this embodiment, a porous material comprising an elastomer matrix comprises a porosity of, e.g., at least 40% of the total volume of an elastomer matrix, at least 50% of the total volume of an elastomer matrix, at least 60% of the total volume of an elastomer matrix, at least 70% of the total volume of an elastomer matrix, at least 80% of the total volume of an elastomer matrix, at least 90% of the total volume of an elastomer matrix, at least 95% of the total volume of an elastomer matrix, or at least 97% of the total volume of an elastomer matrix. In yet other aspects of this embodiment, a porous material comprising an elastomer matrix comprises a porosity of, e.g., at most 40% of the total volume of an elastomer matrix, at most 50% of the total volume of an elastomer matrix, at most 60% of the total volume of an elastomer matrix, at most 70% of the total volume of an elastomer matrix, at most 80% of the total volume of an elastomer matrix, at most 90% of the total volume of an elastomer matrix, at most 95% of the total volume of an elastomer matrix, or at most 97% of the total volume of an elastomer matrix. In yet other aspects of this embodiment, a porous material comprising an elastomer matrix comprises a porosity of, e.g., about 40% to about 97% of the total volume of an elastomer matrix, about 50% to about 97% of the total volume of an elastomer matrix, about 60% to about 97% of the total volume of an elastomer matrix, about 70% to about 97% of the total volume of an elastomer matrix, about 80% to about 97% of the total volume of an elastomer matrix, about 90% to about 97% of the total volume of an elastomer matrix, about 40% to about 95% of the total volume of an elastomer matrix, about 50% to about 95% of the total volume of an elastomer matrix, about 60% to about 95% of the total volume of an elastomer matrix, about 70% to about 95% of the total volume of an elastomer matrix, about 80% to about 95% of the total volume of an elastomer matrix, about 90% to about 95% of the total volume of an elastomer matrix, about 40% to about 90% of the total volume of an elastomer matrix, about 50% to about 90% of the total volume of an elastomer matrix, about 60% to about 90% of the total volume of an elastomer matrix, about 70% to about 90% of the total volume of an elastomer matrix, or about 80% to about 90% of the total volume of an elastomer matrix.

The present specification discloses, in part, a porous material comprising an elastomer matrix defining an array of interconnected pores having a mean open pore value and/or a mean closed pore value that is sufficient to allow tissue growth into the array of interconnected pores as disclosed herein. As used herein, the term “mean open pore value” or “mean open pore” refers to the average number of pores that are connected to at least one other pore present in the elastomer matrix. As used herein, the term “mean closed pore value” or “mean closed pore” refers to the average number of pores that are not connected to any other pores present in the elastomer matrix.

Thus, in an embodiment, a porous material comprising an elastomer matrix defining an array of interconnected pores has a mean open pore value sufficient to allow tissue growth into the array of interconnected pores. In aspects of this embodiment, a porous material comprising an elastomer matrix has a mean open pore value of, e.g., about 70%, about 75%, about 80%, about 85%, about 90%, about 95%, or about 97%. In other aspects of this embodiment, a porous material comprising an elastomer matrix comprises a mean open pore value of, e.g., at least 70%, at least 75%, at least 80%, at least 85%, at least 90%, at least 95%, or at least 97%. In yet other aspects of this embodiment, a porous material comprising an elastomer matrix has a mean open pore value of, e.g., at most 70%, at most 75%, at most 80%, at most 85%, at most 90%, at most 95%, or at most 97%. In still aspects of this embodiment, a porous material comprising an elastomer matrix has a mean open pore value of, e.g., about 70% to about 90%, about 75% to about 90%, about 80% to about 90%, about 85% to about 90%, about 70% to about 95%, about 75% to about 95%, about 80% to about 95%, about 85% to about 95%, about 90% to about 95%, about 70% to about 97%, about 75% to about 97%, about 80% to about 97%, about 85% to about 97%, or about 90% to about 97%.

In another embodiment, a porous material comprising an elastomer matrix defining an array of interconnected pores has a mean closed pore value sufficient to allow tissue growth into the array of interconnected pores. In aspects of this embodiment, a porous material comprising an elastomer matrix has a mean closed pore value of, e.g., about 5%, about 10%, about 15%, or about 20%. In other aspects of this embodiment, a porous material comprising an elastomer matrix has a mean closed pore value of, e.g., about 5% or less, about 10% or less, about 15% or less, or about 20% or less. In yet other aspects of this embodiment, a porous material comprising an elastomer matrix has a mean closed pore value of, e.g., about 5% to about 10%, about 5% to about 15%, or about 5% to about 20%.

The present specification discloses, in part, a porous material comprising an elastomer matrix defining an array of interconnected pores having a void space that is sufficient to allow tissue growth into the array of interconnected pores. As such, the void space should support aspects of tissue growth such as, e.g., cell migration, cell proliferation, cell differentiation, nutrient exchange, and/or waste removal. As used herein, the term “void space” refers to actual or physical space in a porous material comprising an elastomer matrix. As such, the total volume of a porous material comprising an elastomer matrix disclosed herein is based upon the elastomer space and the void space.

Thus, in an embodiment, an elastomer matrix defining an array of interconnected pores has a void volume sufficient to allow tissue growth into the array of interconnected pores. In aspects of this embodiment, a porous material comprising an elastomer matrix comprises a void space of, e.g., about 50% of the total volume of an elastomer matrix, about 60% of the total volume of an elastomer matrix, about 70% of the total volume of an elastomer matrix, about 80% of the total volume of an elastomer matrix, about 90% of the total volume of an elastomer matrix, about 95% of the total volume of an elastomer matrix, or about 97% of the total volume of an elastomer matrix. In other aspects of this embodiment, a porous material comprising an elastomer matrix comprises a void space of, e.g., at least 50% of the total volume of an elastomer matrix, at least 60% of the total volume of an elastomer matrix, at least 70% of the total volume of an elastomer matrix, at least 80% of the total volume of an elastomer matrix, at least 90% of the total volume of an elastomer matrix, at least 95% of the total volume of an elastomer matrix, or at least 97% of the total volume of an elastomer matrix. In yet other aspects of this embodiment, a porous material comprising an elastomer matrix comprises a void space of, e.g., at most 50% of the total volume of an elastomer matrix, at most 60% of the total volume of an elastomer matrix, at most 70% of the total volume of an elastomer matrix, at most 80% of the total volume of an elastomer matrix, at most 90% of the total volume of an elastomer matrix, at most 95% of the total volume of an elastomer matrix, or at most 97% of the total volume of an elastomer matrix. In yet other aspects of this embodiment, a porous material comprising an elastomer matrix comprises a void space of, e.g., about 50% to about 97% of the total volume of an elastomer matrix, about 60% to about 97% of the total volume of an elastomer matrix, about 70% to about 97% of the total volume of an elastomer matrix, about 80% to about 97% of the total volume of an elastomer matrix, about 90% to about 97% of the total volume of an elastomer matrix, about 50% to about 95% of the total volume of an elastomer matrix, about 60% to about 95% of the total volume of an elastomer matrix, about 70% to about 95% of the total volume of an elastomer matrix, about 80% to about 95% of the total volume of an elastomer matrix, about 90% to about 95% of the total volume of an elastomer matrix, about 50% to about 90% of the total volume of an elastomer matrix, about 60% to about 90% of the total volume of an elastomer matrix, about 70% to about 90% of the total volume of an elastomer matrix, or about 80% to about 90% of the total volume of an elastomer matrix.

### Example 1

### Capsule Thickness and Collagen Fiber Alignment

In order to measure the thickness and alignment of collagen fibers in periprosthetic or capsules formed, disks (1 cm in diameter) of various porous materials were implanted subcutaneously in Sprague-Dawley rats using standard procedures. The materials tested were taken from commercially available implants or experimentally produced as follows: Smooth 1, a material having a smooth surface (NATRELLE®, Allergan, Inc., Irvine, Calif.); Smooth 2, a material having a smooth surface (MEMORYGEL®, Mentor, Inc., Santa Barbara, Calif.); Textured 1, a material having a closed-cell textured surface produced from a lost-salt method (BIOCELL®, Allergan, Inc., Irvine, Calif.); Textured 2, a material having a closed-cell textured surface produced from an imprinting method (SILTEX®, Mentor, Inc., Santa Barbara, Calif.); Textured 3, a material having a closed-cell textured surface produced from either an imprinting or gas foam method (SILIMED®, Sientra, Inc., Santa Barbara, Calif.); Textured 4, a material having a closed-cell textured surface produced from an imprinting method (Perouse Plastie, Mentor, Inc., Santa Barbara, Calif.); Textured 5, a material having an open-cell polyurethane surface; Textured 6, a non-polyurethane material having an open-cell textured surface in accordance with one embodiment of the present invention. Samples were harvested at 6 weeks, fixed in formalin, and processed to produce paraffin blocks. The paraffin blocks were sectioned using a microtome at 2 μm thickness and stained with hematoxylin and eosin (H&E).

Capsules were characterized by measuring the thickness and disorganization of the capsule formed over the porous materials. Capsule thickness was measured by acquiring 2 representative 20× images of the H&E stained materials and measuring the thickness of the capsule at 3 points in the image. Capsule collagen fiber alignment was evaluated by acquiring 3 representative 20× images of the H&E stained materials, and then drawing a reference vector tangent to the implant surface, as well as, drawing vectors along collagen fibers within the capsule. The angle of each vector relative to the reference vector was then measured, and the standard deviation of the angles was calculated, where greater standard deviations reflected a higher degree of disorganization. All image analysis calculations were performed on the Nikon Elements Advanced Research software.

All thickness and collagen fiber alignment measurements were acquired blinded and each measurement was normalized to the data obtained from Textured 1 material. For the thickness data collected, a one-way ANOVA was run to determine significant effects (p<0.05). If there were any statistically significant effects from the ANOVA analysis, the Tukey's post-hoc test was run for multiple comparisons at α=0.05. For the fiber alignment data collected, a Levene's Test for Equal Variance was used to determine whether there was a statistically significant difference in disorganization between experimental groups (p<0.05). Between individual groups, the criteria for non-significance were overlap of confidence intervals (95%), adjusted for the number of groups.

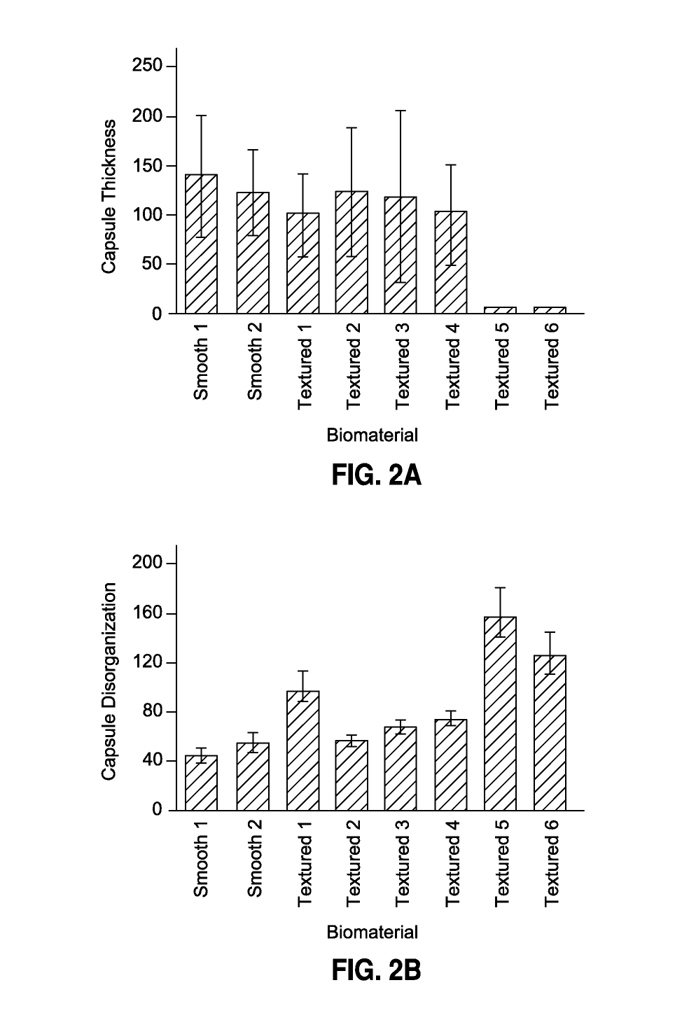

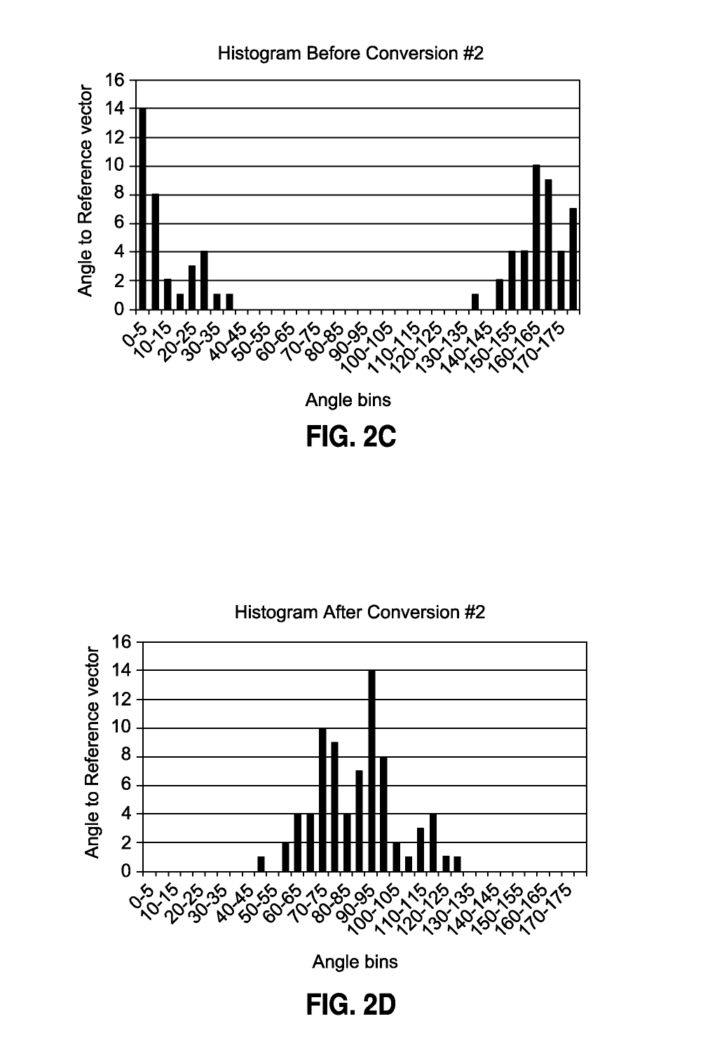

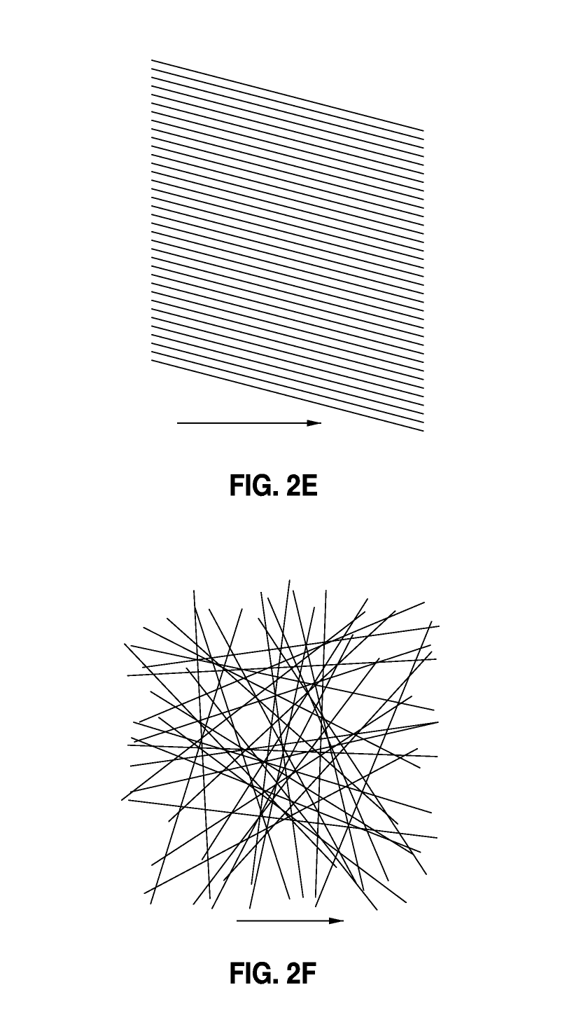

The capsule or periprosthetic thicknesses and collagen fiber alignment, normalized to the Texture 1 material within each respective study, are shown in FIGS. 2A and 2B. Smooth Texture 1 and 2 materials, and Textures 1-4 materials (having closed-cell texture) exhibited pronounced capsule formation, and the capsules formed were of equivalent thicknesses of about 100 μm to about 140 μm. Texture 5-6 materials exhibited minimal capsule formation with capsules formed having a thickness of less than 10 μm. With respect to capsule organization, it was found that Texture 1 material resulted in a capsule that was less aligned than Smooth 1 and 2 and Texture 2-4 materials (FIG. 2B). Texture 5 and 6 materials demonstrated extensive tissue ingrowth that was interconnected through the pores and collagen fibers were significantly less aligned with the tangential vector representing the surface of the implanted material 50% of fibers were not parallel to implant surface tangential vector) than Smooth 1 and 2 and Texture 1-4 materials. These findings show that Smooth 1 and 2 materials (smooth surface) and Textures 1-4 materials (closed-cell textured surfaces) resulted in a capsule with predominantly align collagen fibers. Textures 5-6 materials (open-cell textured surfaces), in contrast, induce significant ingrowth that can eliminate capsule and disorganize the tissue at the material-tissue interface.

### Example 2

### Alignment of Collagen Fibers Analysis

In accordance with one aspect of the invention, circumferential alignment of collagen fibers in periprosthetic tissue (e.g. capsular tissue) is measured by vector analysis. That is, the alignment of fibers around the implant or the extent to which the fibers are parallel to the overall surface of the implant.

A reference vector is drawn parallel to the tissue-device interface on a 20× magnification image of a haematoxylin & eosin (H&E) stained section of the tissue. Twenty five additional vectors are drawn at random along the collagen fibers and the angles relative to the reference vector are recorded. This is repeated twice for a total of 3 images and 75 vector measurements. The recorded angles represent the difference in the direction of the fibers compared to the surface of the implant. If all fibers are parallel, all angles will be either 0 degrees or 180 degrees (the vectors drawn on parallel fibers will either point in the same direction as the reference vector, producing an angle of 0 degrees, or the vectors will point in the opposite direction to the reference vector, producing an angle of 180 degrees). If none of the fibers are parallel, angles will be equally distributed across all measures from 0 degrees to 180 degrees. Vector angles greater than 180° are converted to between 0° and 180° by subtracting 180°. A second mathematical conversion is then applied to produce an even distribution by adding 90° to any angle less than 90°, and subtracting 90° from any angle greater than 90°. The effect of this conversion can be seen in FIGS. 2C and 2D which show the effect of the second angle conversion. Angle measurements are grouped into bins of 5° increments for graphical representation.

The variance in angle distribution is used as a measure of alignment disruption. The greater the alignment, the smaller the variance will be. A perfectly aligned sample will have a variance of zero (FIG. 2E) All angles for a perfectly aligned sample would fall into a single 5° bin on a histogram.

A completely random sample will have a high variance (FIG. 2F) in the range of 45-60.

Capsules from smooth implants have a very small variance, a range of about 14 units, with a confidence interval of ±2 units. Vectors drawn along the fibers are fairly aligned with the reference vector (implant surface).

Tissue from a Biocell™ implant presents with a variance of approximately 31 units, with a confidence interval of ±4 units. Fibers surrounding a Biocell™ implant are less aligned with the implant surface than those fibers surrounding a smooth implant, producing greater variance in angle measurement.

Tissue from an exemplary open cell implant of an embodiment of the present invention presents with a variance of approximately 39 units, with a confidence interval of ±6 units. This indicates a higher variation in angle measurements, suggesting a greater disruption of fiber alignment relative to the implant surface than both Biocell™ or smooth implants.

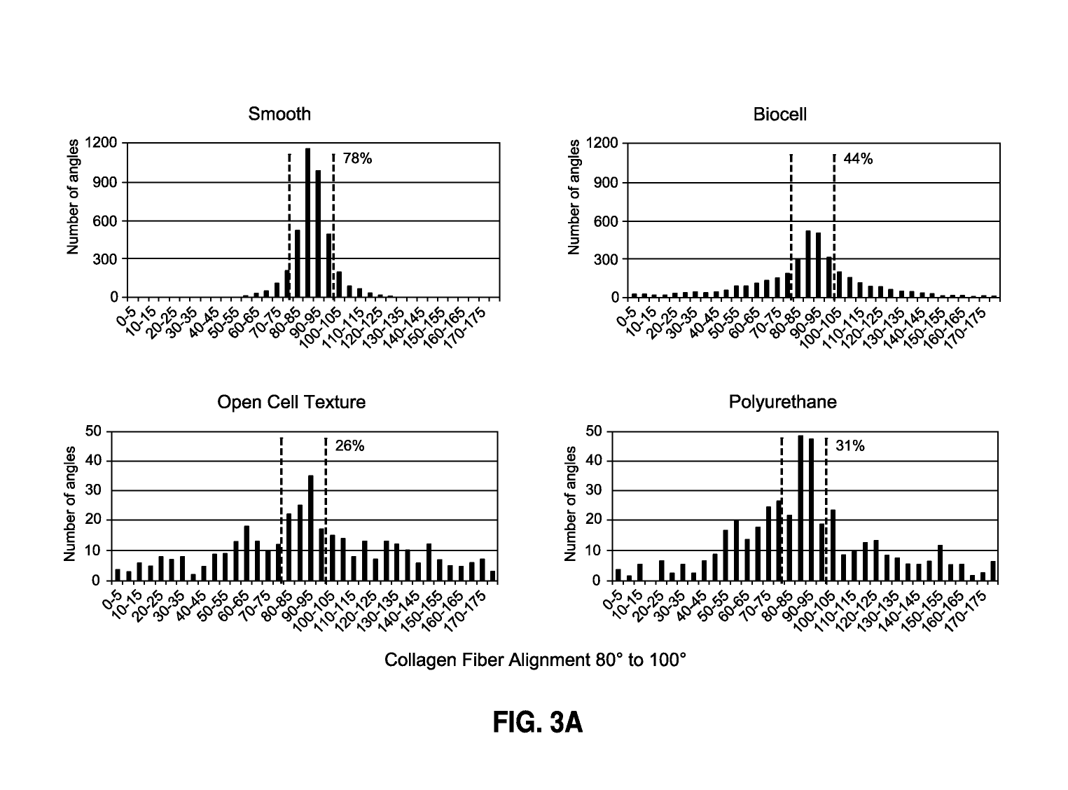

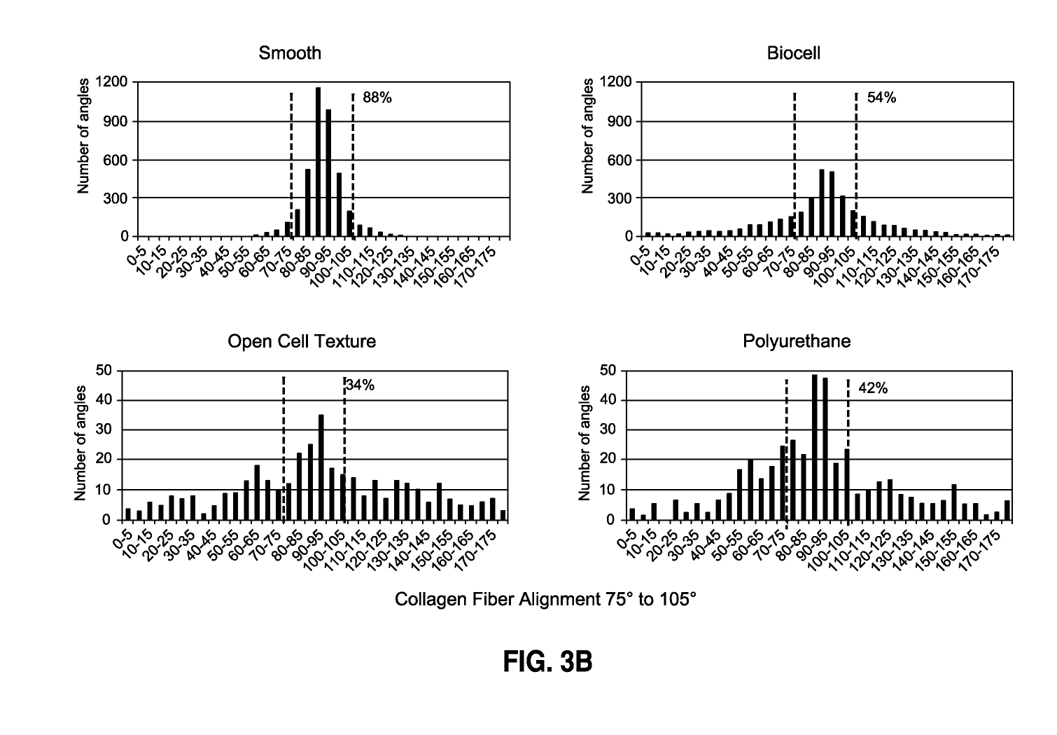

FIG. 3A-3B show histogram representations of collagen fiber alignment assays described in this specification for different test materials.

### Example 3

### Tissue Attachment

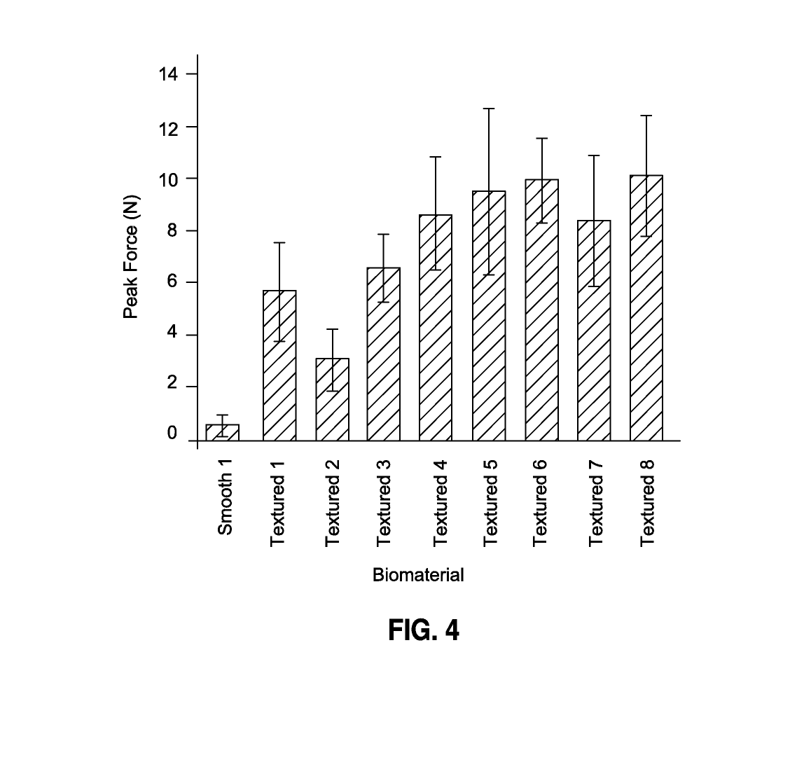

In order to evaluate the effect of texture on tissue attachment or integration into porous materials, strips of various material were implanted subcutaneously in a Sprague-Dawley rat using standard procedures. The materials tested were taken from commercially available implants or experimentally produced as follows: Smooth 1, n=38, a material having a smooth surface (NATRELLE®, Allergan, Inc., Irvine, Calif.); Textured 1, n=64, a material having a closed-cell textured surface produced from a lost-salt method (BIOCELL®, Allergan, Inc., Irvine, Calif.); Textured 2, n=6, a material having a closed-cell textured surface produced from an imprinting method (SILTEX®, Mentor, Inc., Santa Barbara, Calif.); Textured 3, n=6, a material in accordance with the present invention having an inverse foam polyurethane-polyethylene glycol surface; Textured 4, n=45, a material in accordance with the present invention having an inverse foam polyurethane-polyethylene glycol surface; Textured 5, n=45, a material having an open-cell polyurethane surface; Textured 6, n=6, a material having an open-cell polyurethane surface; Textured 7, n=6, a material in accordance with the present invention having an open-cell textured surface of 0.8 mm; Textured 8, n=6, a material in accordance with the present invention having an open-cell textured surface of 1.5 mm. Samples were harvested at 4 weeks, and tissue was pulled from the test strip on a mechanical tester with a pullout speed of 2 mm/second. Tissue integration strength was measured as the peak force required to separate the implant from the surrounding tissue. A one-way ANOVA was run to determine significant effects (p<0.05). If there were any statistically significant effects from the ANOVA analysis, the Tukey's post-hoc test was run for multiple comparisons at α=0.05. Results are shown in FIG. 4.

Smooth 1 material showed little resistance to separation, as there were no significant protrusions above a micro-scale and had minimal drag on the surrounding tissue. Textured 1 and 2 materials (PRIOR ART closed-cell textured surfaces) exhibited limited amount of tissue interaction and showed greater resistance to separation than Smooth 1. Textured 3 and 4 materials (inverse foam textured surface) and Textures 5-8 materials (open-cell textured surfaces of the present invention) degree of resistance to tissue separation from the material with all textures in accordance with the present invention having an open-cell textured surface requiring an average force of greater than 6 Newtons.

### Example 4

### Capsule Stiffness

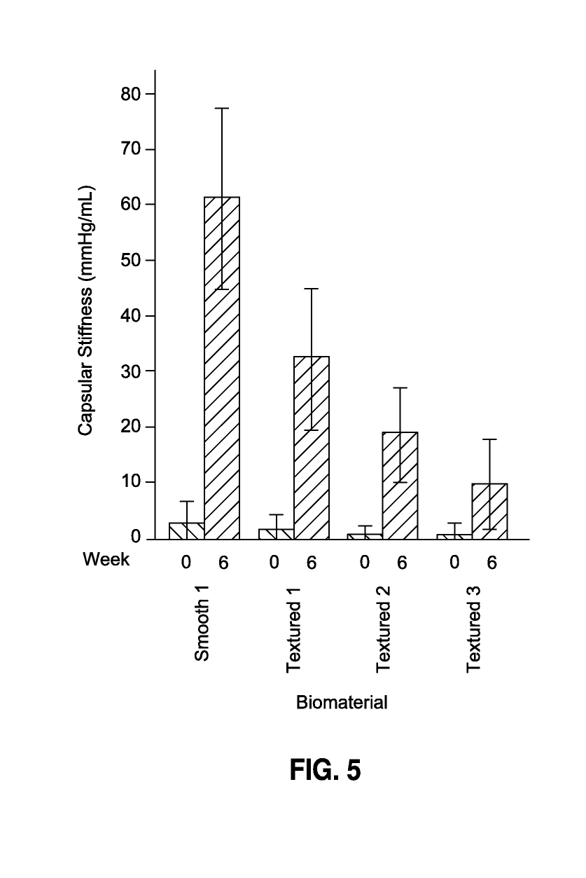

In order to evaluate stiffness of capsules/ingrowth formed over porous materials applied to soft fluid filled implants, 7 mL miniature tissue expanders comprising silicone material of various textures were implanted subcutaneously in a Sprague-Dawley rat using standard procedures. The materials tested were taken from commercially available implants or experimentally produced as follows: Smooth 1, a material having a smooth surface (NATRELLE®, Allergan, Inc., Irvine, Calif.); Textured 1, a material having a closed-cell textured surface produced from a lost-salt method (BIOCELL®, Allergan, Inc., Irvine, Calif.); Textured 2, a material having an open-cell textured surface of 0.8 mm according to embodiments of the present invention; Textured 3, a material having an open-cell textured surface of 1.5 mm produced according to embodiments of the present invention. At time 0 (immediately post-implantation) and at 6 weeks, saline was incrementally added to each expander, and the resulting pressure exerted on and by the expander at each step was measured with a digital manometer. Stiffness was calculated by fitting a trend-line to the linear region of the pressure-volume curve and measuring the slope of the line. Increases in the stiffness of the capsule/ingrowth were reflected by increases in the slope. To account for expander-to-expander variability, each stiffness measurement was normalized to the stiffness of the expander itself. A one-way ANOVA was run to determine significant effects (p<0.05). If there were any statistically significant effects from the ANOVA analysis, the Tukey's post-hoc test was run for multiple comparisons at α=0.05. Results are shown in FIG. 5.

Capsules formed over Smooth 1 material expander showed the greatest stiffness after 6 weeks. Textured 1 material expander (closed-cell textured surface) showed lower stiffness than Smooth 1 material expander but greater stiffness than the Textured 2 and 3 material expanders (open-cell textured surface). This data demonstrated that closed-cell materials resulted in capsules that were stiffer than those that result from open-cell materials of the present invention.

### Example 5

### Capsule Response

In order to identify preferable morphological and physical characteristics of implantable materials, disks (1 cm in diameter) of various biocompatible materials were implanted subcutaneously in Sprague-Dawley rats using standard procedures and the response to such implantation in terms of capsule formation was determined.

### Example 6

### Textured Breast Implant of the Present Invention For Breast Reconstruction

A breast implant having an interconnected porous silicone textured surface is surgically implanted in a 35 year old woman, using standard procedures, following a mastectomy of the left breast. A porous silicone texture covers substantially the entire implant has the following characteristics: a thickness or depth of about 1 mm, a porosity of about 80% to about 88%, a mean pore size of about 480 μm, an mean pore interconnection size of about 110 μm to about 140 μm, a mean ratio of interconnections per pore of between about 4 to about interconnections per pore. One year after the implantation and without extenuating circumstances such as infection or trauma, the patient's left breast retains a Baker grade of 2 or less and remains soft and there are no clinical indications that the breast is developing capsule contracture. Five years later, the breast is still soft and does not experience any hardening or contracture related to the textured implant and not the result of other extenuating clinical circumstances.

### Example 7

### Textured Breast Implant of the Present Invention for Breast Augmentation

In this example, silicone gel filled breast implants having a textured silicone surface in accordance with the present invention, are implanted in both breasts of a 57 year old woman for breast augmentation using standard surgical procedures. The textured surface of the implants have the following characteristics: a pore opening of about 300 to 470 um and 2 to 8 interconnections between pores size of about 90 to about 130 μm, and a surface pore density of about 5.5 per mm².

Two years following the implantation, the breasts of the woman remain soft and show no or minimal signs of contracture.

Accordingly, in one aspect of the invention, a method for treating a patient desiring a mammary prosthesis is provided, the method comprising implanting the mammary prosthesis in the patient, the mammary prosthesis having a textured surface, the textured surface not comprising a polyurethane, wherein within a period of time of about six months after the implanting step, a minimal or no circumferential aligned collagen capsule has formed in proximity to the textured surface, and the capsule has a thickness of less than about 500 microns, for example, less than about 250 microns. The thickness of the capsule is determined by measuring the circumferential aligned collagen capsule thickness at three spaced apart locations on the capsule and averaging the measurements In some embodiments, less than about 50% of collagen fibers associated with the periprosthetic tissue are parallel to the circumferential surface of the mammary prosthesis. In some embodiments, the textured surface is made substantially entirely of silicone elastomer.

In another aspect of the invention, a method for treating a patient in need of a mammary prosthesis is provided wherein the method comprises implanting the mammary prosthesis in the patient, the mammary prosthesis comprising a textured surface as described elsewhere herein, wherein the textured surface is a material made substantially entirely of silicone, and the textured surface does not comprise resorbable polyurethane, wherein within a period of time of about six months after the implanting, the periprosthetic tissue in proximity to the textured surface, has (i) an aligned collagen fiber capsule thickness of less than about 500 microns, for example, less than about 250 microns, as determined using a three points measurement system; and (ii) less than about 50% of collagen fibers of the periprosthetic tissue adjacent to the surface of the implant are parallel to the circumferential plane, or major planar surface, of the implant. Mammary prostheses made with such textured surfaces are also provided. In yet another aspect of the invention, an elastomeric member useful as a component of a breast implant is provided wherein the member comprises a non-polyurethane, open cell material which will result in an aligned collagen circumferential capsule formation of less than about 50 μm thickness after a period of about 6 months after implantation of the material in a breast of a human being, the material having an average pore size of about 300 μm to about 500 μm and an interconnection size of about 150 μm to about 300 μm, and the material being structured such that it encourages substantial tissue ingrowth, minimal capsular formation and/or substantial tissue adhesion.

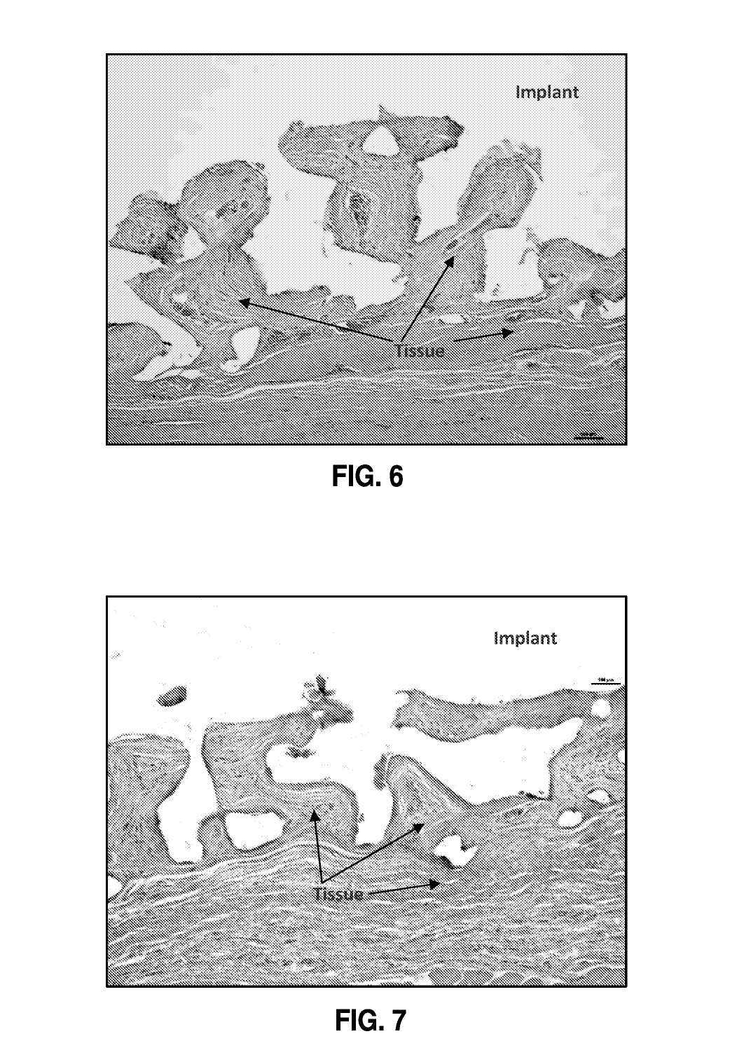

FIGS. 6-11 show images of a periprosthetic tissue adjacent various textured implants in animal studies.

More specifically, FIGS. 6 and 7 show tissue surrounding a textured implant in accordance with the present invention, 6 months after implantation in a sheep (FIG. 6), and after 6 weeks in rat (FIG. 7). The tissue has integrated into the surface texture of the device, as seen in the large tissue projections. No capsular tissue (continuous, circumferential band of parallel collagen fibers) was detected.

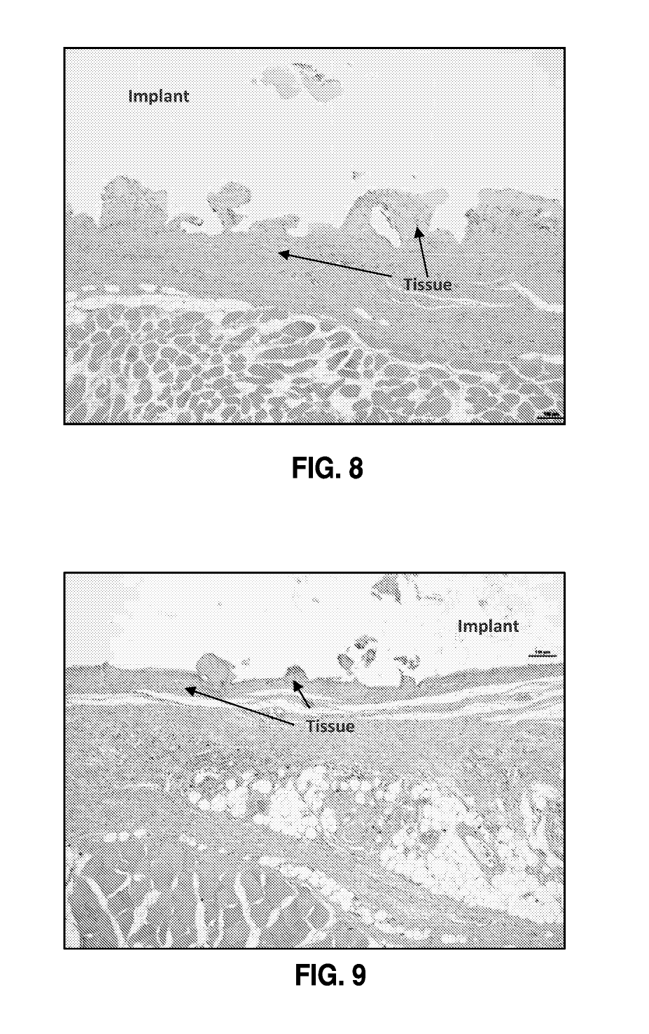

FIGS. 8 and 9, are, respectively images of tissue surrounding a PRIOR ART “closed cell” (Biocell™) implant after 6 months in sheep and after 6 weeks in rat. The tissue has grown into the open cell surface texture of the device, providing an interaction with the surface texture without integration through interconnecting pores. The periprosthetic tissue shows some capsular tissue formation (continuous, circumferential band of parallel fibers).

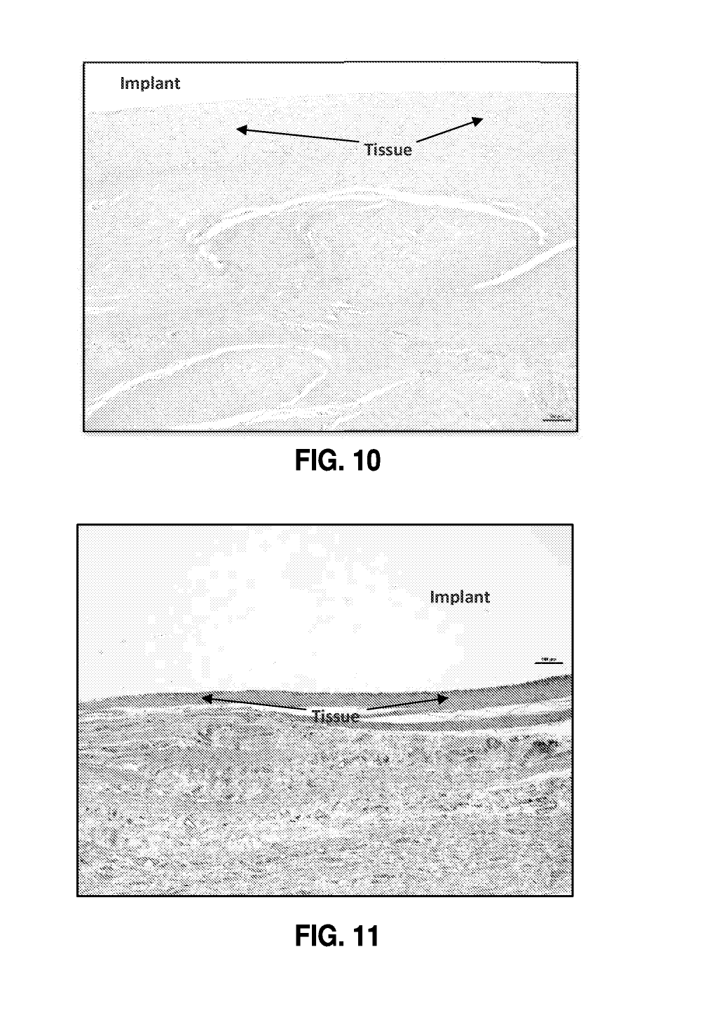

FIGS. 10 and 11 are, respectively, images of tissue surrounding a PRIOR ART smooth (non-textured) implant after 6 months in a sheep and after 6 weeks in rat. The periprosthetic tissue shows no integration or interaction with the surface texture. The tissue is highly characteristic of capsular tissue (continuous, circumferential band of parallel fibers).

### Example 8

### Comparison of Material Degradation In Vivo

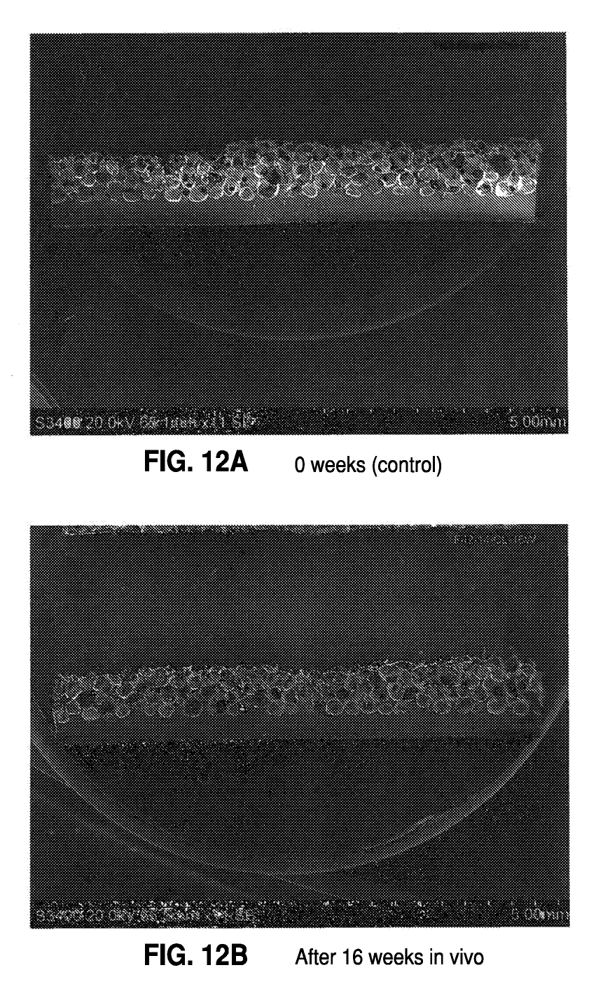

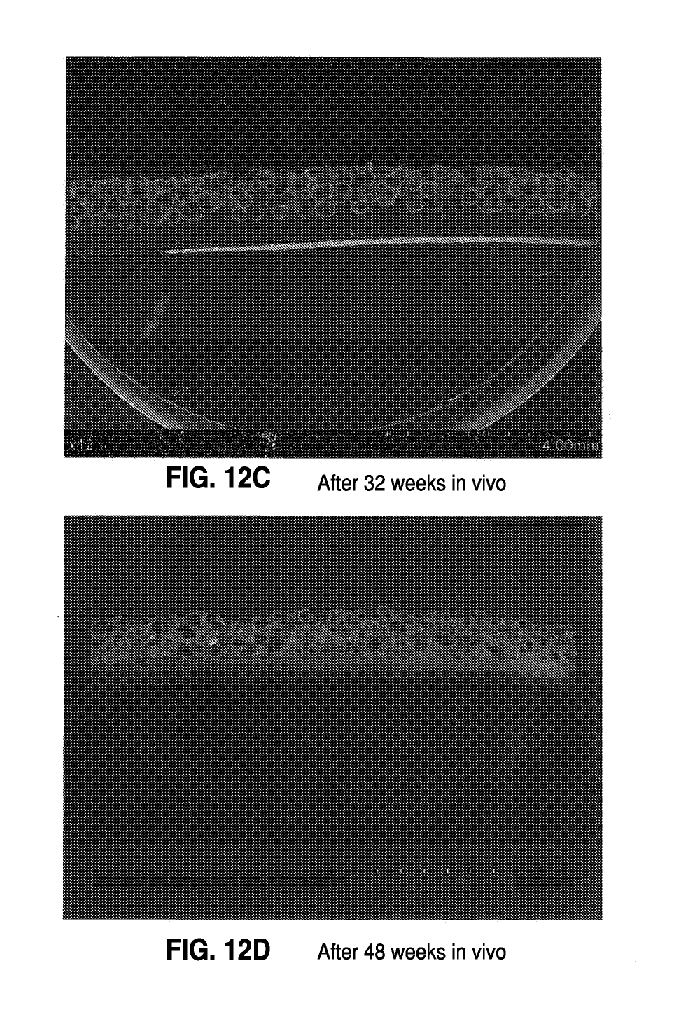

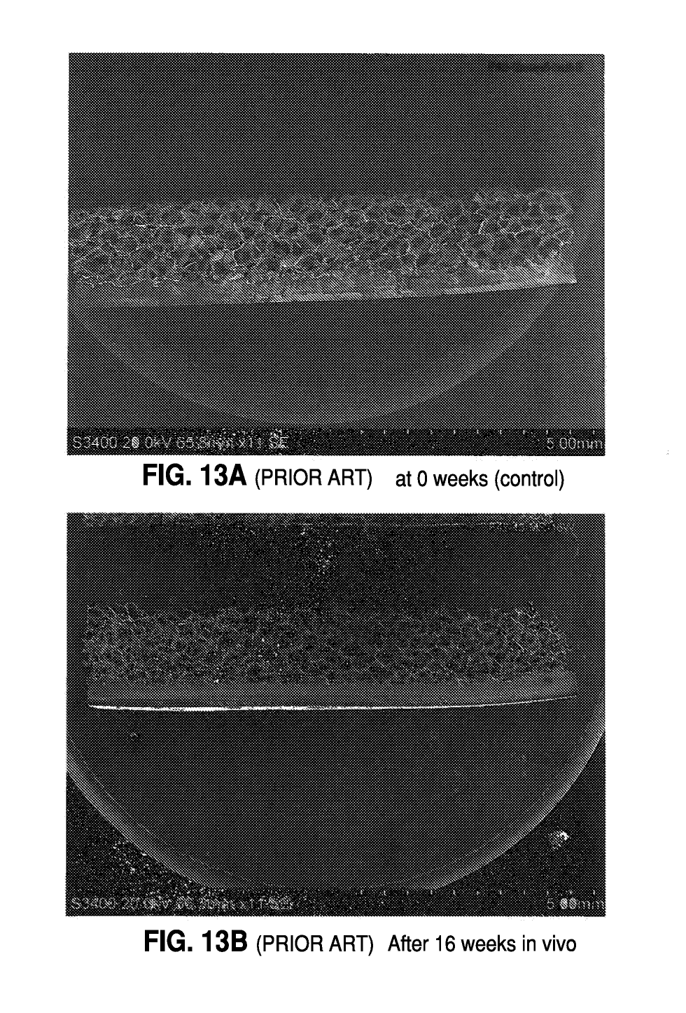

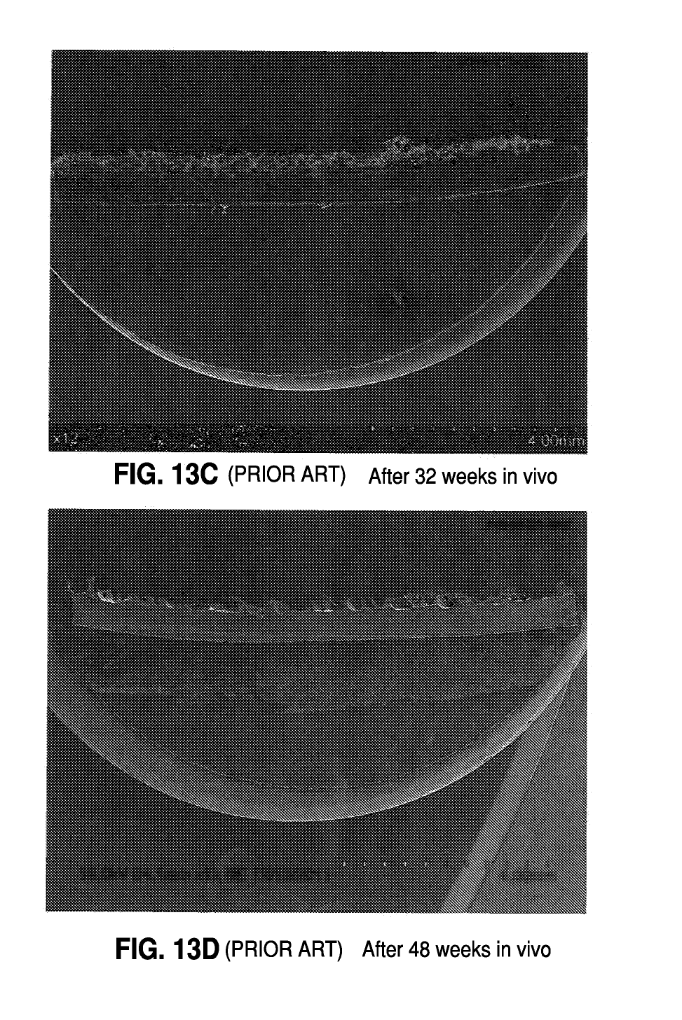

Turning now to FIGS. 12A through 13D, each image shows approximately one-half of a 10 mm disk of material in cross section at 11× or 12× magnification.

Briefly, a 10 mm disk was cut from a shell of a breast implant device, implanted subcutaneously in rats and after 16, or 48 weeks, the implant was removed and the tissue surrounding the implant was enzymatically digested. The material was rinsed in alcohol and dried. Each disk was weighed and then cut in half. One half was prepared for scanning electron microscopy and imaged at 11× or 12× at the cross section to visualize the depth of the surface texture. The depth of the surface texture was measured. A 10 mm disk that had not been implanted was also weighed, cut in half, prepped and imaged at 11× or 12× as a control.

Referring to FIGS. 12A-12D which are images of a silicone based, porous material in accordance with the present invention, there is no visible degradation of the material. When these samples from 16 weeks, 32 weeks and 48 weeks were compared to a control sample (0 weeks, FIG. 12A), the thickness of the material surface texture, that is, the silicone-based open pore structure arising out of the silicon shell, had shown no appreciable changes. Furthermore, there was no statistically significant difference in thickness of the silicone texture. There was a small change in the absolute value, which may represent compression, not degradation. In addition, weight data indicates no change in weight of this material. The number, size and pores of the texture are visually similar across time, and the complexity or interconnectivity of the pores remains comparable.

The images for PRIOR ART polyurethane implants (FIGS. 13A-13D) can be seen to be substantially different over time. When comparing the 16 weeks sample to the control (0 weeks, FIG. 13A), the thickness of the surface texture was similar, but visible thinning of the individual fibers or “struts” of the polyurethane material was visible. At 32 weeks there is a significant difference in surface texture thickness, where half or more of the polyurethane applied to the surface of the silicone shell had degraded. The remaining struts have thinned, and the overall standard polyurethane structure is lost. At 48 weeks the surface texture had disappeared almost entirely. There were few visible struts left, which create no more than a single layer of texture complexity. For these samples, there was also a significant decrease across time in both thickness of surface texture and weight, indicating the surface texture may have separated from the underlying silicone shell.

In another aspect of the invention, an assay is provided for determining or quantifying collagen fiber alignment in periprosthetic tissue in a mammal.

The method generally comprises the steps of: (a) obtaining a sample to be analyzed, the sample comprising periprosthetic tissue and adjacent material that has been explanted from a mammal and (b) obtaining at least one section of the sample, for example, at least two sections, or at least three sections or more, of the sample, the section including both periprosthetic tissue and at least a portion of the explanted adjacent material, the section being between about 5 microns to about 10 microns. The method further comprises the steps of (c) staining, for example, using hematoxylin and eosin (H&E), the at least one section to reveal collagen fibers in the tissue and (d) providing a magnified image of the stained section, for example, at about 20× magnification. The method further comprises the steps of (e) placing a reference vector on the magnified image, the reference vector being parallel to the major plane of the material at a tissue and material interface; (f) placing a plurality of alignment vectors on the image, the alignment vectors being indicative of alignment of said collagen fibers revealed on the image; (g) recording an angle of each of the alignment vectors with respect to the reference vector; and (h) calculating a variance of the recorded angles to thereby quantify a collagen fiber alignment of the sample of periprosthetic tissue.

The method may further comprise recording includes grouping the angles into bins, to create a histogram, for example, a histogram such as shown in FIGS. 3A and 3B. The bins may be bins representing 5 degree increments, for example, as shown.

In some embodiments, the plurality of alignment vectors comprises at least 25 alignment vectors, for example, at least 25 alignment vectors per obtained section. For example, the step of obtaining at least one section comprises obtaining three such sections and the plurality of alignment vectors comprises about 75 alignment vectors, with about 25 alignment vectors per each of the three such sections.

Further, the method may further comprise the step of performing a mathematical conversion on the alignment vectors such that 180° is subtracted from each alignment vector that has a measurement of greater than 180°. In some embodiments, the step of performing a mathematical conversion further comprises adding 90° to each alignment vector having an angle of less than 90°, and subtracting 90° from each alignment vector having an angle greater than 90°. The alignment angles may then be represented, after the mathematical conversion, on a histogram showing 5 degree bins versus number of angles falling within each 5 degree bin.

Further, the method may comprise the step of representing the alignment angles on a histogram includes calculating the number of angles falling within a range around degrees. The calculation may include, for example, determining the number of angles in each bin in a range of about degrees to about 110 degrees, a range about 75 degrees to about 105 degrees, or a range of between about 80 degrees and 100 degrees. FIGS. 3A and 3B show histograms for samples of periprosthetic tissue adjacent different textures/types of explanted materials using the presently described methods. FIGS. 3A and 3B show the percentage of angles of collagen fibers, of the total number of angles, falling within the 80 degree to 100 degree range, and falling within the 75 degree to 105 degree range, respectively.

A control test may be provided, wherein the control test comprises performing steps (a) through (h) using a control sample comprising a smooth (untextured) material and determining whether number of angles falling in a range of 80 to 100 degrees is at least 60%.

For example, in FIG. 3A, the number of angles falling within 80 degrees to 100 degrees was calculated to be about 78%, indicating a high percentage of collagen fibers being substantially aligned parallel to the implant surface.

In certain embodiments, “non-aligned” collagen fibers of periprosthetic tissue are defined as those collagen fibers of the tissue that have angles falling outside of the 80 degree to 100 degree range, when the periprosthetic tissue is tested as described in Examples 1 and 3, and as illustrated, for example, in FIG. 3A.

In a specific embodiment, an implantable, non-resorbable material is provided comprising a textured surface defined by open interconnected cavities wherein the material satisfies the criteria of, when tested using the assay described herein in Examples 1 and 2, results in the formation of periprosthetic tissue defined by: greater than 22% of collagen fibers being not aligned with the implant surface when non-aligned collagen fibers are calculated as those having angles falling outside of the 80 degree to 100 degree range.

In yet another embodiment, an implantable, non-resorbable material is provided comprising a textured surface defined by open interconnected cavities wherein the material satisfies the criteria of, when tested using the assay described herein in Examples 1 and 3, results in the formation of periprosthetic tissue defined by: an adhesion force of greater than 6 Newtons.

In still another specific embodiment, an implantable material is provided comprising a textured surface defined by open interconnected cavities wherein the material satisfies the criteria of, when tested using the assay described herein in Examples 1 and 2, results in the formation of periprosthetic tissue defined by: greater than 56% of collagen fibers being not aligned parallel to the implant surface (for example, the number of collagen fiber angles falling outside of the 80 degree to 100 degree range) and when tested using the assay described herein in Examples 1 and 3, results in the formation of periprosthetic tissue defined by: an adhesion force of greater than 6 Newtons.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A method of augmenting or reconstructing soft tissue of a patient, the method comprising the step of:
implanting into the patient, a prosthesis comprising a non-resorbable, open cell material characterized in that, when a one centimeter disc of a test material identical to the material is implanted subcutaneously in a Sprague Dawley rat using standard procedures, periprosthetic tissue forms adjacent the test material, and at least six weeks following the subcutaneous implantation, the periprosthetic tissue has a characteristic that at least 22% of collagen fibers of the periprosthetic tissue are non-aligned with respect to a major planar surface defined by the test material.

2. The method of claim 1 wherein said periprosthetic tissue, at the period of time of about six weeks from the implanting step, has the characteristic that at least 25% of collagen fibers of the periprosthetic tissue are non-aligned with respect to a major planar surface defined by the test material.

3. The method of claim 1 wherein said periprosthetic tissue, at the period of time of about six weeks from the implanting step, has the characteristic that at least 50% of collagen fibers of the periprosthetic tissue are non-aligned with respect to a major planar surface defined by the test material.

4. The method of claim 1 wherein said periprosthetic tissue, at the period of time of about six weeks from the implanting step, has the characteristic that at least 56% of collagen fibers of the periprosthetic tissue are non-aligned with respect to a major planar surface defined by the test material.

5. The method of claim 1 wherein the prosthesis is a breast prosthesis and the step of implanting into a patient comprising implanting the prosthesis into a breast of the patient.

6. The method of claim 1 wherein the method is a breast augmentation procedure.

7. The method of claim 1 wherein the method is a breast reconstruction procedure.

8. The method of claim 1 wherein the prosthesis is a fluid-filled implant.

9. The method of claim 1 wherein the prosthesis is a fluid-filled breast implant.

10. A method of augmenting or reconstructing a breast of a patient, the method comprising the step of:
implanting into the patient, a prosthesis comprising a non-resorbable, open cell material characterized in that, when a one centimeter disc of a test material identical to the material is implanted subcutaneously in a Sprague Dawley rat using standard procedures, periprosthetic tissue forms adjacent the material, and at least six weeks following the subcutaneous implantation, the periprosthetic tissue has an adherence force of at least 6 Newtons.

11. The method of claim 10 wherein said periprosthetic tissue, at the period of time of about six weeks from the implanting step, has the further characteristic that at least 56% of collagen fibers of the periprosthetic tissue are non-aligned with respect to a major planar surface defined by the test material.

12. The method of claim 10 wherein the prosthesis is a breast prosthesis and the step of implanting into a patient comprising implanting the prosthesis into a breast of the patient.

13. The method of claim 8 wherein the method is a breast augmentation procedure.

14. The method of claim 10 wherein the method is a breast reconstruction procedure.

15. The method of claim 10 wherein the prosthesis is a fluid-filled implant.

16. The method of claim 1 wherein the prosthesis is a fluid-filled breast implant.
